(19) United States
(12) Patent Application Publication
(10) Pub. No.: US 2008/0154179 A1
(43) Pub. Date: Jun. 26, 2008

(54) AGENT DELIVERY SYSTEM

(75) Inventors: Hal C. Cantor, West Bloomfield, MI (US); Scott A. Cantor, West Bloomfield, MI (US); Kenneth H. Swartz, St. Clair Shores, MI (US); Robert Hower, Farmington Hills, MI (US)

Correspondence Address: BROOKS KUSHMAN P.C., 1000 TOWN CENTER, TWENTY-SECOND FLOOR, SOUTHFIELD, MI, 48075, US

(73) Assignee: TRANSDERMAL PATENTS COMPANY, LLC, Troy, MI (US)

(21) Appl. No.: 11/949,721

(22) Filed: Dec. 3, 2007

Related U.S. Application Data
(63) Continuation-in-part of application No. PCT/US2006/021762, filed on Jun. 5, 2006.
Application No. PCT/US2006/021762 is a continuation-in-part of application No. PCT/US2006/021761, filed on Jun. 5, 2006.
Application No. PCT/US2006/021761 is a continuation-in-part of application No. PCT/US2006/021763, filed on Jun. 5, 2006.
(60) Provisional application No. 60/687,262, filed on Jun. 3, 2005.
(60) Provisional application No. 60/687,262, filed on Jun. 3, 2005.
(60) Provisional application No. 60/687,262, filed on Jun. 3, 2005.

(51) Int. Cl. A61N 1/30

(52) U.S. Cl. 604/20

(57) ABSTRACT

An automated, controllable, and affixable pulsatile agent delivery system having an automated controller for controlling the delivery of drug to a patient, an agent delivery reservoir containing an agent operatively connected to the automated controller, a reservoir controller operatively connected to the automated controller and the reservoir for controlling the delivery of agent to a patient, and a feedback control operatively connected to the automated controller for providing feedback with regard to the drug requirements of the patient. A method of delivering an agent to a patient in need of the same by administering the above agent delivery system to a patient, determining an amount of agent needed for the patient, and affecting administration of the agent to the patient via the agent delivery system.

### CROSS-REFERENCE TO RELATED APPLICATIONS

This application is a continuation-in-part of International Patent Application Nos. PCT/US2006/021761, filed 5 Jun. 2006, published in English, which claims the benefit of provisional patent application Ser. No. 60/687,262, filed Jun. 3, 2005; PCT/US2006/021762, filed 5 Jun. 2006, published in English which claims the benefit of provisional patent application Ser. No. 60/687,262, filed Jun. 3, 2005; and PCT/US2006/021763, filed 5 Jun. 2006; and which claims the benefit of provisional patent application Ser. No. 60/687,262, filed Jun. 3, 2005. The disclosures of these applications are hereby incorporated by reference in their entireties.

### BACKGROUND OF THE INVENTION

1. Field of the Invention

Generally, the present invention provides an agent delivery system. More specifically, the present invention provides an automated system for delivery of drugs or compounds.

2. Description of the Related Art

The skin functions as the primary barrier to the transdermal penetration of materials into the body and represents the body's major resistance to the transdermal delivery of beneficial agents such as drugs. To date, efforts have concentrated on reducing the physical resistance of the skin or enhancing the permeability of the skin to facilitate the delivery of drugs by passive diffusion. Various methods of increasing the rate of transdermal drug flux have been attempted, most notably by using chemical flux enhancers.

The delivery of drugs through the skin provides many advantages. Primarily, such a means of delivery is a comfortable, convenient and noninvasive way of administering drugs. The variable rates of absorption and metabolism encountered in oral treatment are avoided, and other inherent inconveniences, e.g., gastrointestinal irritation and the like are eliminated as well. Transdermal drug delivery also makes possible a high degree of control over blood concentrations of any particular drug.

However, many drugs are not suitable for passive transdermal drug delivery because of their size, ionic charge characteristics and hydrophilicity. One method of achieving transdermal administration of such drugs is the use of electrical current to actively transport drugs into the body through intact skin. The method of the present invention relates to such iontophoresis, which is an example of such an administration technique.

Herein the terms “electrotransport”, “iontophoresis”, and “iontophoretic” are used to refer to the delivery of pharmaceutically active agents through a body surface by means of an applied electromotive force to an agent-containing reservoir. The agent may be delivered by electromigration, electroporation, electroosmosis or any combination thereof. Electroosmosis has also been referred to as electrohydrokinesis, electro-convection, and electrically induced osmosis. In general, electroosmosis of a species into a tissue results from the migration of solvent in which the species is contained, as a result of the application of electromotive force to the therapeutic species reservoir, which results in solvent flow induced by electromigration of other ionic species. During the electrotransport process, certain modifications or alterations of the skin may occur such as the formation of transiently existing pores in the skin, also referred to as “electroporation”. Any electrically assisted transport of species enhanced by modifications or alterations of the body surface (e.g., formation of pores in the skin) are also included in the term “electrotransport” as used herein. Thus, as used herein, the terms “electrotransport”, “iontophoresis” and “iontophoretic” refer to (a) the delivery of charged drugs or agents by electromigration, (b) the delivery of uncharged drugs or agents by the process of electroosmosis, (c) the delivery of charged or uncharged drugs by electroporation, (d) the delivery of charged drugs or agents by the combined processes of electromigration and electroosmosis, and/or (e) the delivery of a mixture of charged and uncharged drugs or agents by the combined processes of electromigration and electroosmosis.

Systems for delivering ionized drugs through the skin have been known for some time. British Patent Specification No. 410,009 (1934) describes an iontophoretic delivery device that overcame one of the disadvantages of the early devices, namely, the need to immobilize the patient near a source of electric current. The device was made by forming, from the electrodes and the material containing the drug to be delivered, a galvanic cell which itself produced the current necessary for iontophoretic delivery. This device allowed the patient to move around during drug delivery and thus required substantially less interference with the patient's daily activities than previous iontophoretic delivery systems.

In present day electrotransport devices, at least two electrodes are used simultaneously. Both of these electrodes are disposed so as to be in intimate electrical contact with some portion of the skin of the body. One electrode, called the active or donor electrode, is the electrode from which the drug is delivered into the body. The other electrode, called the counter or return electrode, serves to close the electrical circuit through the body. In conjunction with the patient's skin, the circuit is completed by connection of the electrodes to a source of electrical energy, e.g., a battery, and usually to circuitry capable of controlling current passing through the device. If the ionic substance to be driven into the body is positively charged, then the positive electrode (the anode) can be the active electrode and the negative electrode (the cathode) serves as the counter electrode, completing the circuit. If the ionic substance to be delivered is negatively charged, then the cathodic electrode can be the active electrode and the anodic electrode can be the counter electrode.

All electrotransport agent delivery devices utilize an electrical circuit to electrically connect the power source (e.g., a battery) and the electrodes. In very simple devices such as those disclosed by Ariura et al in U.S. Pat. No. 4,474,570, the “circuit” is merely an electrically conductive wire used to connect the battery to an electrode. Other devices use a variety of electrical components to control the amplitude, polarity, timing, waveform shape, etc. of the electric current supplied by the power source. See, for example, U.S. Pat. No. 5,047,007 issued to McNichols et al.

Existing electrotransport devices additionally require a reservoir or source of the pharmaceutically active agent that is to be delivered or introduced into the body. Such drug reservoirs are connected to an electrode, i.e., an anode or a cathode, of the electrotransport device to provide a fixed or renewable source of one or more desired species or agents. A reservoir would include a reservoir matrix or gel that contains the agent and a reservoir housing which physically contains the reservoir matrix or gel. In addition to the drug reservoir, an electrolyte-containing counter reservoir is generally placed between the counter electrode and the body surface. Typically, the electrolyte within the counter reservoir is a buffered saline solution and does not contain a therapeutic agent. In early electrotransport devices, the donor and counter reservoirs were made of materials such as paper (e.g., filter paper), cotton wadding, fabrics and/or sponges that could easily absorb the drug-containing and electrolyte-containing solutions. In more recent years however the use of such reservoir matrix materials has given way to the use of hydrogels composed of natural or synthetic hydrophilic polymers. See for example, U.S. Pat. No. 4,383,529, to Webster, and U.S. Pat. No. 6,039,977, to Venkatraman. Such hydrophilic polymeric reservoirs are preferred from a number of standpoints, including the ease with which they can be manufactured, the uniform properties and characteristics of synthetic hydrophilic polymers, their ability to quickly absorb aqueous drug and electrolyte solutions, and the ease with which these materials can be handled during manufacturing. Such gel materials can be manufactured to have a solid, non-flowable characteristic. Thus, the reservoirs can be manufactured having a predetermined size and geometry.

Generally, the geometry of a reservoir can be described in terms of three parameters: (1) the average cross-sectional area of the reservoir (“A_{RES}”), defined as the arithmetic mean of reservoir cross-sectional areas measured at a number of different distances from and parallel to the body surface; (2) the average thickness of the reservoir; and (3) the body surface contact area (“A_{BODY}”). References to reservoir housing configuration and the above parameters include not only the parameters of the physical reservoir housing, but also include the physical parameters of the reservoir gel or matrix as well.

Electrotransport drug delivery devices having a reusable controller for use with more than one drug-containing unit have been described. The drug-containing unit can be disconnected from the controller when the drug becomes depleted and a fresh drug-containing unit can then be connected to the controller. The drug-containing unit includes the reservoir housing, the reservoir matrix, and associated physical and electrical elements that enable the unit to be removably connected, both mechanically and electrically to the controller. In this way, the relatively more expensive hardware components of the device (e.g., the batteries, the light-emitting diodes, the circuit hardware, etc.) can be contained in the reusable controller. The relatively less expensive donor reservoir and counter reservoir may be contained in the single use, disposable drug containing unit. See, U.S. Pat. No. 5,320,597, to Sage et al.; U.S. Pat. Nos. 5,358,483 and 5,135,479, both to Sibalis. Electrotransport devices having a reusable electronic controller with single use/disposable drug units have also been proposed for electrotransport systems comprised of a single controller adapted to be used with a plurality of different disposable drug units. For example, WO 96/38198, to Johnson et al., discloses the use of such reusable electrotransport controllers which can be connected to drug units for delivering the same drug, but at different dosing levels, (e.g., a high dose drug unit and a low dose drug unit) which can be connected to the same electrotransport controller. Although these systems go far in reducing the overall cost of transdermal electrotransport drug delivery, further cost reductions are needed in order to make this mode of drug delivery more competitive with traditional delivery methods such as by disposable syringe.

To date, commercial transdermal iontophoretic drug delivery devices (e.g., the Phoresor, sold by Iomed, Inc. of Salt Lake City, Utah; the Dupel Iontophoresis System sold by Empi, Inc. of St. Paul, Minn.; the Webster Sweat Inducer, model 3600, sold by Wescor, Inc. of Logan, Utah) have generally utilized a desk-top electrical power supply unit and a pair of skin contacting electrodes. The donor electrode contains a drug solution while the counter electrode contains a solution of a biocompatible electrolyte salt. The “satellite” electrodes are connected to the electrical power supply unit by long (e.g., 12 meters) electrically conductive wires or cables. Examples of desktop electrical power supply units which use “satellite” electrode assemblies are disclosed in Jacobsen et al U.S. Pat. No. 4,141,359; U.S. Pat. No. 5,006,108, to LaPrade et al; and U.S. Pat. No. 5,254,081, to Maurer.

More recently, small self-contained electrotransport delivery devices adapted to be worn on the skin, sometimes unobtrusively under clothing, for extended periods of time have been proposed. The electrical components in such miniaturized iontophoretic drug delivery devices are also preferably miniaturized, and may be in the form of either integrated circuits (i.e., microchips) or small printed circuits. Electronic components, such as batteries, resistors, pulse generators, capacitors, etc. are electrically connected to form an electronic circuit that controls the amplitude, polarity, timing waveform shape, etc. of the electric current supplied by the power source. Such small self-contained electrotransport delivery devices are disclosed for example in Tapper U.S. Pat. No. 5,224,927; Haak et al U.S. Pat. No. 5,203,768; Sibalis et al U.S. Pat. No. 5,224,928; and Haynes et al U.S. Pat. No. 5,246,418. One concern, particularly with small self-contained electrotransport delivery devices that are manufactured with the drug to be delivered already in them, is the potential loss in efficacy after a long period of device storage. In an electrotransport device using batteries and other electronic components, all of the components have various shelf lives. If it is known, for example, that the batteries used to power these small delivery devices gradually degrade, and the drug delivery rate may go off specification. It would be advantageous to have a means to limit the active life of the delivery device for a certain period of time (e.g., months) after device manufacture in order to prevent this potential loss in device efficacy.

Application of therapeutic drugs, whether by electrotransport or more traditional (e.g., oral) dosing, can sometimes cause unwanted reactions in certain patients. These reactions can take many forms, including change in heart rate, change in body temperature, sweating, shaking and the like. It would be advantageous to automatically and permanently disable an electrotransport drug delivery device upon encountering such “unwanted” reactions.

Therefore, there is a need for a near-continuous non-invasive device for monitoring composition levels with automated, near-continuous infusion of appropriate amounts of an appropriate compound in the effort to achieve normal, i.e. non-diseased, states at all times. It would be desirable to have such devices available in a condition in which the abuse potential of the device is reduced without diminishing the intended therapeutic efficacy of the device or the abusable substance to be administered.

### SUMMARY OF THE INVENTION

The present invention is a pulsatile agent delivery system is a portable iontophoretic device to be attached to the skin. The device is based upon the micro-electro-mechanical system (MEMS) and/or complementary metal oxide semiconductor (CMOS) technology. The device contains two battery-powered electrodes, which send a charged ion across the skin iontophoretically. The battery can be one or more thin film or watch batteries. The battery can be built into the agent delivery system housing or may be integrated into the detachable agent delivery reservoir. This device can also be used in a hospital setting operating on an AC/DC power source. The agent delivery system of the present invention is controlled by an automated controller, which is based on an integrated circuit, which controls the timing and activation of the iontophoretic delivery of the agent from the agent delivery reservoir.

The agent delivery system can be configured to both deliver a therapeutic agent and extract interstitial fluid to analyze agent concentration in the body or monitor a surrogate marker to determine when additional agent is necessary. The device unlike other iontophoretic devices is able to deliver the charge on a pulsed basis rather than continuously. The pulsed delivery may be timed to: optimize drug concentration requirements; reduce drug waste; reduce the potential for antibiotic drug resistance; and, developing a tolerance to therapeutic agents. The agent delivery system can vary the pulse to increase the interval between doses or reduce the amount of agent delivered over time. The “ramp down” characteristic is a novel way to wean a patient off an addictive drug.

The sampling chamber used to analyze interstitial fluid can be placed directly adjacent to the skin. The agent delivery reservoir containing the agent:polymer mixture is attached to a biocompatible membrane which is in turn is covered with a biocompatible adhesive and attached to the skin. The adhesive is chosen to retain the device in place for the duration of the treatment period. (i.e. 24 hrs or 4 weeks). The agent delivery system may also be adapted to provide a physical attachment device, i.e. a wristband or a strap. The agent delivery reservoir can be a fixed reservoir or a detachable reservoir to facilitate changes in agents or agent concentration.

An agent can be either a hydrophobic or hydrophilic molecule prepared in a polymer such as poly(etheleneoxide) (PEO) or DMSO and stored in an agent delivery reservoir. The agent and polymer are stored in an agent reservoir. The agent:polymer mixture is configured in a single reservoir or several layered agents, in distinct rings, in a single reservoir. The single or layered agents are located over the agent delivery electrode for iontophoretic delivery. The layered agents provide the ability to include delivery enhancing agents and healing agents to reduce skin irritability.

The ability to sample, test, monitor and provide feedback to the automated controller is provided by the feedback control unit. The present invention allows configuration of the agent delivery system to be a stand-alone agent delivery system, a stand-alone feedback control unit or an agent delivery system used in conjunction with the feedback control unit. The feedback control unit operates by reverse iontophoresis, electro-osmosis and/or electroporation to extract interstitial fluid from the skin into a reaction chamber reservoir.

Testing or monitoring of a specific agent is operated by an independent feedback-automated controller, which may be linked to the delivery-automated controller to provide input regarding the timing of the next delivery of the therapeutic agent. The testing reagents are stored in reagent storage reservoirs. The testing reagents are delivered to the reaction chamber reservoir by microfluidic conduits, which are polymer based and attached to a silicon chip, which is operated by microfluidic pressure driven pumps that in one embodiment utilize hydrogel or hydrocarbons that are heated to swell and compress at fixed intervals to drive the reagents to the reaction chamber.

Testing is conducted by chemical, immunologic (ELISA) or chromogenic methods and requires a detection system. Detecting agents using a single electrochemical sensor or an array of electrochemical sensors accomplish monitoring. The sensors send a signal based on the reaction to the agent delivery system automated controller to administer or not administer the agent.

The sensor information may also be sent to a data storage unit. The agent delivery system can be configured to store data including the interval of delivery of agent and feedback monitoring results. These data can be communicated or transmitted to an external computer by either an integrated USB port or a wireless based technology such as “Bluetooth”. The agent delivery system may also be configured to accept an externally communicated signal to reprogram the interval for pulsed delivery of the agent.

### BRIEF DESCRIPTION OF THE DRAWINGS

Other advantages of the present invention can be readily appreciated as the same becomes better understood by reference to the following detailed description when considered in connection with the accompanying drawings wherein:

FIG. 1A illustrates an embodiment of the present invention of a one-time use device, wherein the device includes a collection chamber and several assaying chambers, and 1B illustrates another embodiment of the present invention of a system, wherein the system includes at least one sensor connected to a remote display system and at least one collection chamber, at least one separation chamber, and at least one sensing chamber in communication with the other chambers through micro-conduits;

FIGS. 2A and 2B show the CAD layout of the chambers wherein two chips constitute the top and bottom of the device;

FIG. 3 shows the complete mask layout;

FIG. 4 shows the cross-section of the assembled chip;

FIG. 5 shows top and bottom pieces of the chamber, mated together;

FIG. 6 shows a thick bead of photoresist material at the corner of the etched;

FIG. 7 shows that the vaporized OP was bubbled through an appropriate buffer solution, causing the OP to dissolve back into the liquid to be assayed;

FIG. 8 is a graph that shows the activity of the enzyme was determined by measuring the change in absorbance (or slope) after one month and two months of storage at −4 C;

FIG. 9 shows that the separation of the enzyme globule from the plastic substrate caused the effective surface area of the immobilized enzyme to increase, enabling more substrate to react with the enzyme;

FIG. 10 shows that there was significant suppression of enzyme activity in the 2P1 immobilized enzyme wells;

FIG. 11 shows the results of a kinetic protocol was created on the photometric micro-titer plate reader to take an absorbance reading at 405 nm every minute for 10 minutes, and compute an average slope;

FIG. 12, show almost identical slopes for control and plasma cholinesterase, confirming the capacity of the BTC substrate to detect cholinesterase activity in plasma;

FIG. 13 shows that acetylcholinesterase from RBC lysate had significant activity (slope=53.6 mOD/min) when the AcTC substrate was used, whereas there was significantly less activity (slope=13.7 mOD/min) for the reaction using the BTC substrate;

FIG. 14 shows the effect of selective inhibition on plasma samples that were treated with quinidine (20 μM), the inhibitory effect was observed only when BTC was used;

FIG. 15 shows the effect of selective inhibition on plasma samples that were treated with quinidine (20 μM), the inhibitory effects of cholinesterase activity with and without quinidine was observed;

FIG. 16 shows that diluted and undiluted plasma showed cholinesterase activity using substrate reagents that were dried and spotted individually;

FIG. 17 shows that the present invention can include a detection chamber that can fit into a conventional 96 well plate and read using a conventional spectrophotometer;

FIG. 18 shows that absorbance increased in a linear manner for the wells containing plasma and also shows that a detectable color change occurred;

FIG. 19 shows the reliability of the sampling and immunoassay analysis and a correlation to literature values, the pre melatonin saliva values were averaged (n=5, MEAN=17.5+/−8.4 pg/mL);

FIG. 20 shows that in normal adults, serum melatonin concentrations are highest during the night (about 60 to 200 pg/mL) and lowest during the day (about 10 to 20 pg/mL) and that these concentrations are well within the melatonin standard curve as determined by amperometry;

FIG. 21 shows a glucose (Sigma, Cat. No. EC No 200-075-1, Lot No. 41 K0184) standard curve that was prepared with concentrations ranging from 50 mg/dL to 400 mg/dL;

FIG. 22 shows that the diode acts as a quarter wave stack, enhancing the signal at certain wavelengths;

FIG. 23 shows that the response of the diodes is linear to the amount of incident power;

FIG. 24 shows optical chemical sensors reproduced on silicon chips by incorporating a photo-diode with an optical membrane on top of the diode;

FIG. 25 is a photomicrograph of the 2 μm sensor array;

FIG. 26 shows a different size sensor array chips bonded in a ceramic carrier;

FIG. 27 shows a schematic of the sensor array;

FIG. 28 shows alternative sensor array configurations;

FIG. 29 shows an inhibition of the ChE activity that was demonstrated in the presence of OP;

FIGS. 30A, 30B, 30C, and 30D show a variety of different support mechanisms located within a chamber of the present invention;

FIGS. 31A, 31B, and 31C show a variety of support mechanism spacing within a chamber of the present invention;

FIGS. 32A and 32B are CAD drawings of a transdermal sampling chamber of the present invention;

FIG. 33 shows a microfluidic system of the present invention;

FIG. 34 shows a microfluidic actuator and microfluidic valve of the microfluidic system of the present invention;

FIG. 35 is a cross-sectional layout of the fluid analyzing device;

FIG. 36 is a cross-sectional layout of the fluid analyzing device with a separation membrane (electrolyte polymer membrane);

FIG. 37 is a cross-sectional view of a system of the present invention including a removable membrane interface chamber;

FIG. 38 is a schematic view of a CAD layout of the fluid analyzing device and the fluid analyzing system, this chip measures 8 mm×4 mm×2 mm, the membrane interface chamber resides underneath the chip;

FIG. 39 is a cross-sectional view of the fluid delivery device with supports;

FIG. 40 is a cross-sectional view of the fluid delivery device, with an electrolyte polymer membrane;

FIG. 41 is a schematic view of the fluid analyzing system on one body portion;

FIG. 42 is a cross-sectional view of the fluid analyzing system on one body portion;

FIG. 43 is a cross-sectional view of the fluid analyzing system on two body portions;

FIG. 44 is a dose-response curve of closed loop delivery vs. standard methods of delivery;

FIG. 45 is a back view of a mock-up of a patch with pulsatile delivery, approximately 2 cm in diameter (the size of a band-aid);

FIG. 46 is a flow chart of a model-based controller;

FIG. 47 illustrates a comparison of lithium delivery methods in hairless mice; and

FIG. 48 illustrates a software interface.

### DETAILED DESCRIPTION OF THE INVENTION

In an embodiment of the present invention, an agent delivery device is provided. The agent delivery device of this embodiment is useful for administering a biologically compatible agent to a patient. The agent delivery device includes an agent delivery reservoir containing the agent to be administered to the patient. An electrolyte receives at least a portion of the agent from the agent delivery reservoir. The electrolyte is mixed with the agent to form an electrolyte-agent mixture that is contained in the reservoir. Moreover, the electrolyte-agent mixture traps the agent until electric current is applied thereto. The device also includes an agent delivery surface in communication with the electrolyte. In a refinement, the agent delivery device includes one or more additional delivery surfaces. The agent delivery surface contacts the patient and delivers agent received from the reservoir to the patent. A controller in communication with the electrolyte-agent mixture provides a series of control pulses to the electrolyte. Each pulse allows the device to administer a portion of the agent to the patient. The series of pulses provides a temporally varying concentration of agent in the patient. In a variation, the electrolyte comprises an iontophoretic electrically conductive material. In a further refinement, the electrolyte is polymeric. The term iontophoretic electrically conductive material means any material that exhibits iontophoretic behaviour.

In a variation of the present embodiment, the temporally varying concentration of agent includes a plurality alternating agent concentration maxima and minima with the maxima and minima differing by a predetermined amount. In a further refinement, the maxima and minima differ by at least 5%, 10%, 20%, 30%, 40% and 50% in order of increasing preference. In some refinements, the temporally varying concentration of agent is matched to the turnover of cell receptors for the agent. In another refinement, the temporally varying concentration of agent is matched to the life-cycle of an invading bacteria or parasite In another refinement, the temporally varying concentration of agent is such that agent concentration maxima in the patient is increased over time. In a further refinement, the series of pulses provide a temporally increasing agent concentration maxima in the patient for a first predetermined time period. In still a further refinement, the series of pulses provides a temporally decreasing agent concentration maxima in the patient for a second predetermined time period that occurs after the second time period. In another refinement, the temporally varying concentration of agent is such that agent concentration maxima in the patient is decreased over time

As set forth above, the agent delivery device of the invention includes a digital controller and a memory accessible to the digital controller. An algorithm for controlling the electrolyte is encoded in the memory such that the algorithm may be executed by the digital controller. In a refinement, one or more intervals of the series of controlled pulses are varied over time via the controlling algorithm. In another refinement, the amplitudes of the series of controlled pulses are varied via the controlling algorithm. In still another variation, the duration or width of the series of controlled pulses is varied via the controlling algorithm.

In another variation, the agent delivery devise further includes a sensor system for determining the concentration of the agent in the patient. In a refinement of this variation, such a sensor system is advantageously used in a feedback loop to the controller. In such a feed back loop, information from the sensor system is used to adjust the concentration of the agent or one more additional agents in the patient.

In another embodiment of the present invention, a method of delivering a biologically compatiable agent to a patent is provided. The method of this embodiment utililzes the agent delivery device set forth above. The method of this embodiment comprises contacting the patient with the agent delivery surface and then operating the controller to administer the agent to the patent.

A number of different compositions may be used for the agent in the present inventions. Examples of such compositions include anti-malarial agents, hormones, antiretroviral drug, antibiotic drugs, antipsychotic drugs (e.g., lithium), addictive agents, chemotherapeutic cancer agent, cosmetic anti-wrinkle agent, naturally occurring or synthetic hydrophilic or hydrophobic agents, analgesic agents, and the like. Specific examples of anti-malarial agents include amodiaquine, artemether, artemisinin, artesunate, atovaquone, cinchonine, cinchonidine, chloroquine, doxycycline, halofantrine, mefloquine, primaquine, pyrimethamine, quinine, quinidine, sulfadoxine, and combinations thereof. Specific examples of hormones include gonadotropin releasing hormones (GnRH), estradiol, progesterone, growth hormone, thyroid stimulating hormone (TSH) prolactin, human parathyroid hormone buserelin, insulin, and combinations thereof. Specific examples of antiretroviral drugs include abacavir, didanosine, indinavir, lamivudine, nevirapine, ritonavir, saquinavir mesylate, zalcitabine, zidovudine, and combinations thereof. Specific examples of antibiotic drugs include ampicillin, azithromycin, doxycycline, erythromycin, penicillin, tetracycline, and combinations thereof. Specific examples of addictive agents include nicotine, morphine, methadone, and combinations thereof. Specific examples of chemotherapeutic cancer agents include Buserelin, Taxol, and combinations thereof. Specific examples of cosmetic anti-wrinkle agents include acollagen, collagen-glycosaminoglycan, polytetrafluoroethylene, poly-L-lactide and poly(ethyleneoxide)-poly(butyleneterephthalate), polyglactin, polyglycolic acid, biosynthetic materials, hydrocolloid-like materials, and combinations thereof. Specific examples of analgesic agents include non-steroidal anti-inflammatory drugs, steroids, COX-1 inhibitors, COX-2 inhibitors, and combinations thereof.

Generally, the present invention provides a completely automated, miniaturized agent delivery system/device 10 capable of delivering different types of agents from or into a minute amount of fluid. The device of the present invention can treat the disease or the physiologic condition. More specifically, the present invention is a micro-electro-mechanical system (MEMS) based device 10 with optionally integrated fluid acquisition or microfluidic system 11 and external monitoring system 44.

The top layer of the skin, the stratum corneum, is the main barrier to drug and molecular transport, however with the help of an electric current, molecules can pass through the skin easier. There are two principal mechanisms by which iontophoresis enhances delivery of an agent across the skin: (a) iontophoresis, in which a charged ion is repelled from an electrode of the same charge, and (b) electroosmosis, the convective movement of solvent that occurs through a charged “pore” in response to the preferential passage of counter-ions when the electric field is applied. Iontophoresis can also be operated in the reverse, wherein applying an electric current across the skin extracts a substance from beneath the skin. For larger molecules, and increased transport, electroporation uses short (100-300 ms) pulses of very high voltage (50-250V) to increase transdermal interstitial fluid transport. This method of drug delivery increases mass transport across the dermal membrane by several orders of magnitude. Electroporation efficiency is dependent on both the duration and amplitude of applied voltage. Short pulses between 4V and 15V have been shown to increase the epidermal conductance, but not the effective pore radii, while longer pulses (on the order of 50 min) have been demonstrated to increase pore radii. This method is compatible with larger molecule transport through the skin, at much higher rates, and it has been demonstrated that 40 Kda molecules can be transported through the skin with this method without any skin enhancers.

The present invention can also be used to detect the presence of various agents and substances as described above. Additionally, the present invention can detect and determine whether exposure to an agent has occurred through the detection of antibody presence and levels thereof. Additionally, the present invention can be used to detect the biological effect of exposure to such various agents and substances as described above.

### Agent Delivery Device

Unlike the prior art systems, the agent delivery device 10 of the present invention allows delivery of hydrophilic as well as hydrophobic molecules, such as antibiotics. The agent delivery device 10 is smaller (less than 2 cm²), less expensive to manufacture, and utilizes an electrolyte polymer to trap the drug in large quantities and release it, as square-wave pulses, only when iontophoretic current is applied. The agent delivery device 10 is fully programmable utilizing on-chip custom complementary metal oxide semiconductor (CMOS) circuitry, thus allowing it to be programmed for any pulse length and frequency regime. Using a programmed algorithm, the timing and duration of each pulse can be changed throughout the treatment to provide the agent delivery pattern sufficient to provide appropriate protection without overdosing, underdosing, creating resistance to the drug, or any of the other known side effects.

Alternatively, the invention as described can also monitor a subject's reaction to various agents and deliver the agent based upon a predetermined level detected by the agent delivery device. This agent delivery device 10 is small and non-invasively monitors interstitial fluids that are in equilibrium with the concentration in blood. The agent delivery device 10 contains a low power micro-fluidic pump for transporting fluid sample to the sensors, micro-fluidic conduits and valves for routing sample and calibration solutions, silver/silver chloride (Ag/AgCl) reference electrodes for electrical stimulation of the skin, microscopic semiconductor sensors to detect ions and chemicals, and electronic circuitry to control the pumps and valves as well as to provide integration with existing data-logging and telemetry systems. FIG. 37 depicts a cross-section of the final device with sampling and sensor chambers, waste reservoir, and three polysilicon heaters with membrane actuators to act as the peristaltic pump.

As mentioned above, the agent delivery device 10 of the present invention utilizes significantly less power than conventional microfluidic devices. It is compatible with standard CMOS fabrication and therefore the controlling circuitry can be integrated onto the substrate. It is calculated that less than 700 μW of power is necessary to achieve a pumping rate of 10 μL/min and that pumping rates of 100 μL/min are achievable with this design. Pumping volumes are accurate to within 5 nL volumes.

The transdermal delivery of drugs, by diffusion through a body surface, offers improvements over more traditional delivery methods, such as subcutaneous injections and oral delivery. Transdermal drug delivery also avoids the hepatic first pass effect encountered with oral drug delivery. Generally the term “transdermal”, when used in reference to drug delivery, broadly encompasses the delivery of an agent through a body surface, such as the skin, mucosa, nails or other body surfaces (e.g., an organ surface) of an animal.

### Pulsatile Agent Delivery Timing

The method of delivering drugs and metabolites to patients using the agent delivery device 10 of the present invention follows normal physiological concentrations patterns, as opposed to super- or pharmaco-physiological concentrations and patterns, the timing of which is based on systemic factors including receptor dynamics, drug clearance, drug half-life, etc. The delivery timing may also be based on feedback via monitoring of the actual delivered molecule (i.e., lithium or nicotine) or by monitoring of a second indicator molecule (i.e., glucose monitoring for insulin administration). This provides “on-demand” delivery of the agent. Further, the “on-demand” delivery of agents/drugs maintains the body loads at the therapeutic level as opposed to the great oscillations present when administered orally or via a bolus injection. The invention provides pulsatile delivery of the agent/drug and continuous “ramp-down” capability, controlled automatically at predetermined intervals, or based upon agent or surrogate marker monitoring. The administration of the agent occurs objectively, without requiring a subjective analysis. This aids in limiting overdosing or creating an addiction to an agent, because the administration is based upon readily ascertainable bodily events that can be tested/analyzed objectively. Since only the necessary amount of agent is being administered, lower amounts of agents can be administered. The end result of the pulsatile agent delivery system are fewer side effects, less drug resistance, less increased tolerance to agents, and through increased patient compliance increasing the number of individuals that are able to benefit from the agents.

### Agent/Polymer Mixtures

Polymer matrix electrolytes have been shown to be ideal for storage and delivery of molecules, such as lithium using iontophoresis. Polymer electrolytes are solid-like materials formed by dispersing a molecule/therapeutic, such as nicotine for cessation of smoking, in a high molecular weight polymer. In essence, the molecule is trapped within the polymer until the application of an electric current. Application of electric current, such as by electrodes causes the porosity of the polymer to increase, hence providing controlled release of a molecule. This technology allows molecular concentrations of nicotine as high as 4M to be incorporated into the matrix. The use of polymer electrolytes to deliver molecules can simplify the agent delivery device considerably since it may eliminate the need for reservoirs and pumps. CMOS circuitry controls the amplitude and duration of the molecule transfer in order to deliver precise amounts of the desired molecule. This may also provide a secondary fail-safe mechanism in case of trauma to the agent delivery device 10′, or failure mode operation since transdermal delivery of the desired molecule can only occur when current is applied.

Polymer electrolytes are ionically conducting polymers that are composed essentially of solutions of ionic salts in heteropolymers, such as poly(ethylene oxide) (PEO). PEO is a semicrystalline solid with a high proportion of crystalline regions distributed in a continuous amorphous phase, which means the PEO is a solid at room temperature (tm=65° C. and Tg=−60° C., thus it has structural integrity) and the PEO chains in the amorphous regions have a sufficient degree of segmental mobility, permitting ion transport. The amount and state of amorphous regions of polymer is therefore crucial to its functioning as a polymer electrolyte, which can be altered by many factors, including the type and amount of added ions (including medicinal drugs) and the method by which the polymer electrolyte is formed.

As its low molecular weight analogs, the poly(ethylene glycol)s, the PEO has minimal adverse reactions to skin (skin irritation and sensitization), as well as a sufficient loading capacity of drug dose. Unlike its low molecular weight analog poly(ethylene glycol), which tends to form liquid or semisolids, PEO forms a solid matrix. The drug delivery property of the polymer electrolyte film for iontophoresis is assessed by checking its AC impedance. PEO-salt complexes can be formed as soft, flexible films with a thickness that can vary from a few micrometers to about 100 micrometers.

Previous studies showed that PEO can incorporate large concentrations (˜4M) of salt, making it eminently suitable as a matrix into which highly potent drugs may be incorporated. Other iontophoretic conducive electrolytes, including DMSO, may be selected by those skilled in the art. The identification of PEO is not meant to be a limitation in selection of a polymer that is compatible with the requirements of the agent delivery system.

### Biocompatible Membrane

The agent delivery device 10 includes a biocompatible body portion 13′ housing a transmembrane fluid capturing chamber 12′ for capturing interstitial fluid through a membrane 60′ and a testing chamber 54′ for detecting molecules in captured interstitial fluid, as shown generally in FIG. 35. The transmembrane fluid capturing chamber 12′ is also described as a membrane interface chamber 12′ because it is situated against and adjacent to a membrane 60′. The membrane 60′ can be skin, a membrane in vitro, or any suitable membrane in/on a body. The agent delivery device 10′ is small, on the order of a few square centimeters or less. The agent delivery device 10′ integrates the circuitry, microfluidic devices, and other elements of the miniaturized agent delivery device 10.

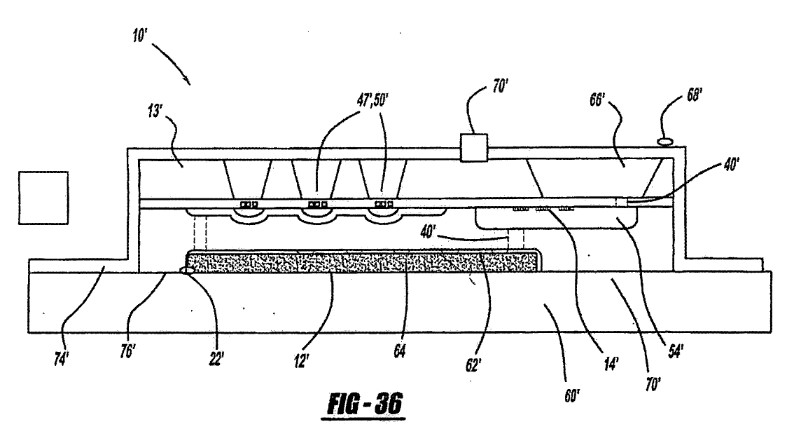

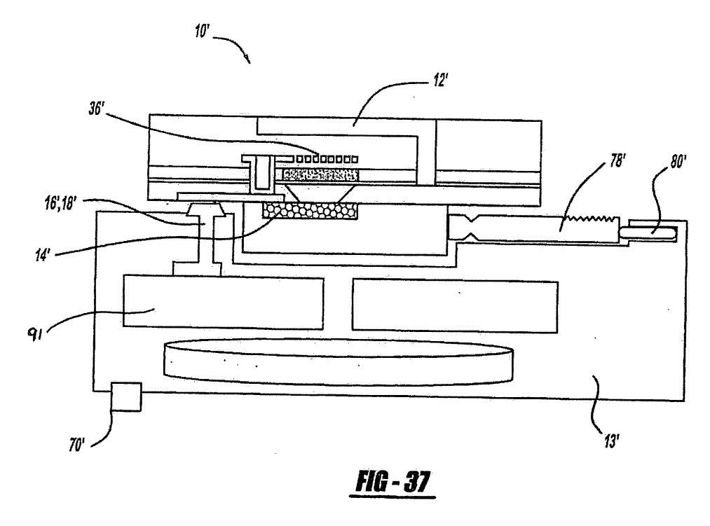

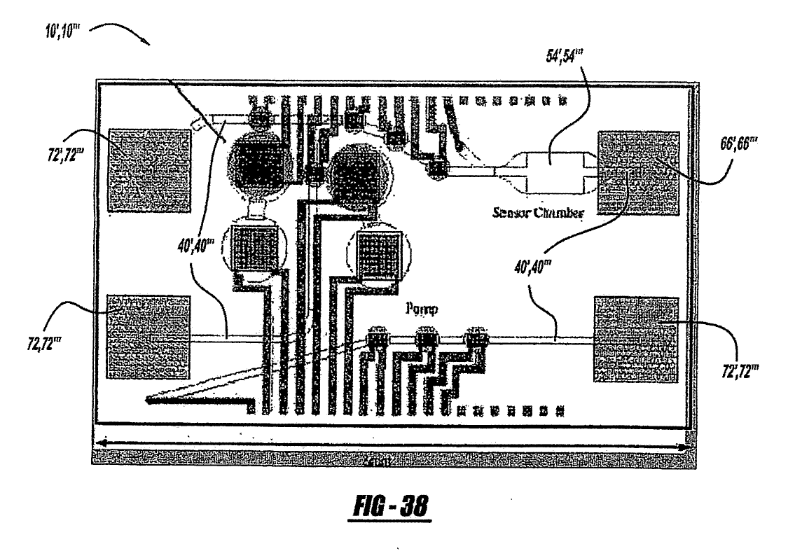

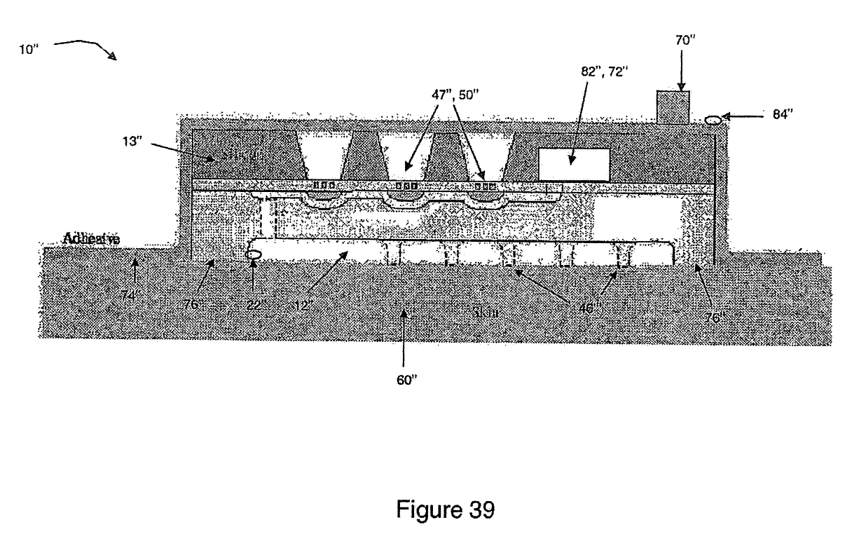

The membrane interface chamber 12′ can include an operatively attached electrode(s) 22′ for performing iontophoresis/electroporation in order to obtain interstitial fluid from the membrane 60′. The base 62′ can also be covered by a at least one separation membrane 64 to maintain a gap or a distance between the base 62′ of the membrane interface chamber 12′ and the membrane 60′, as shown in FIG. 36.

The separation membrane 64 can be any suitable membrane, for example an electrolyte polymer membrane 64. Choice of membrane should be based on whether the molecules to be delivered to or extracted from the skin are hydrophilic or hydrophobic. It is also recommended that when a hydrophobic molecule is to be delivered or extracted two membranes, one hydrophobic layer and one hydrophilic layer, to facilitate transport across the skin.

### Reservoirs

According to the present invention, there is provided an agent delivery device having an agent delivery reservoir containing a polymer and an agent contained within the polymer, wherein the reservoir is capable of pulsatile delivery of the agent.

The molecular delivery apparatus 82″ can be at least one reservoir 72″ operatively attached to the membrane interface chamber 12″ by micro-conduits 40″. Reservoirs may be fixed single use reservoirs or interchangeable reservoirs. The reservoir(s) 72″ can be controlled by microfluidic valves 50″ and microfluidic pumps 47″. Agents are stored in the reservoir 72″ until the need for administration when they are released into the membrane interface chamber 12″ to be administered through the membrane 60″. Other fluids can also be stored in the reservoir 72″, such as wash fluid or any other suitable fluid. Additionally, the agent delivery device 10 of the present invention can include numerous reservoirs 72″. The reservoirs 72″ do not have to all contain the same agent. Instead, adjacent reservoirs 72″ can contain agents that work in concert with one another. For example, one reservoir 72″ can contain the needed agent and the next reservoir 72″ can contain a skin healing agent or chemical enhancer that aids in the delivery of the needed agent. The benefit of such a configuration is a limit in potential skin irritation at the site of agent administration. Alternatively, the reservoir 72″ can be layered with different agents being encapsulated in the layers.

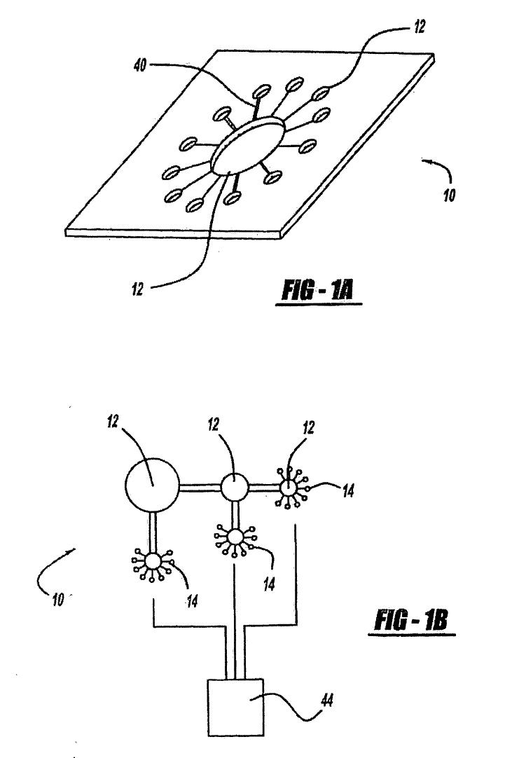

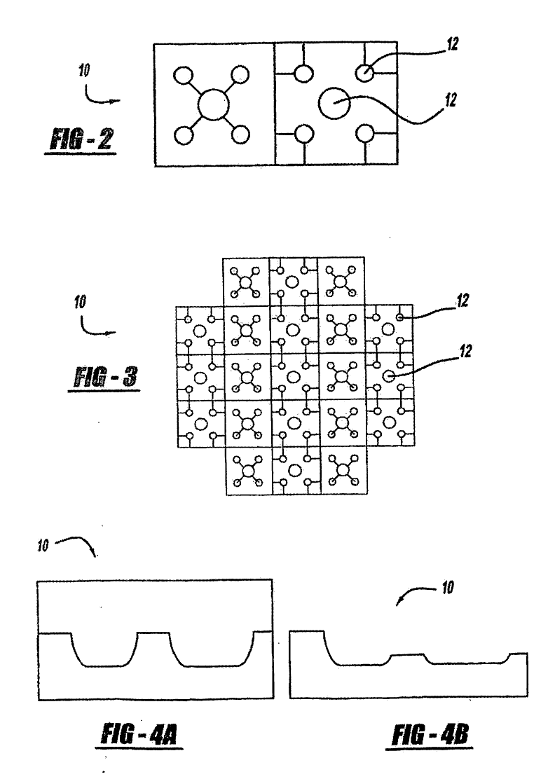

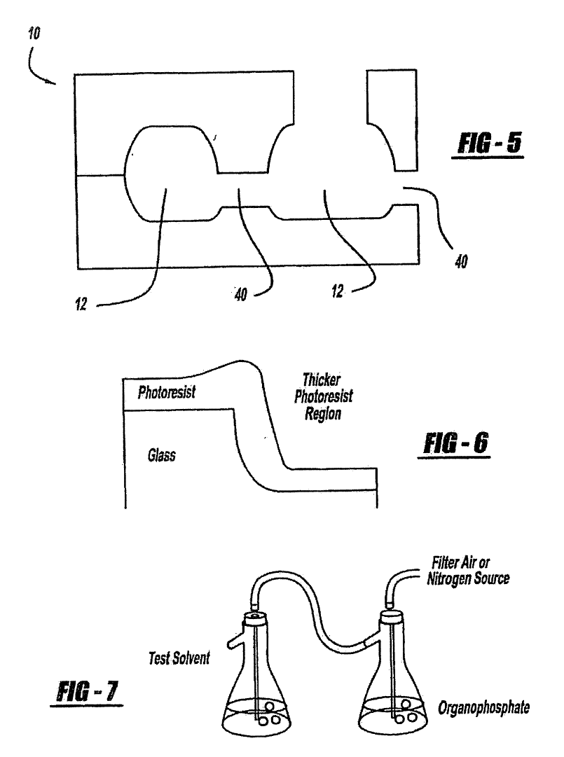

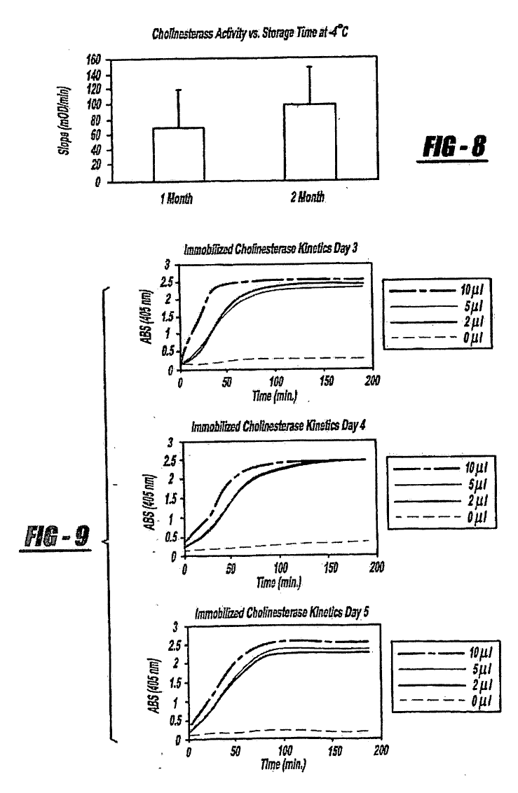

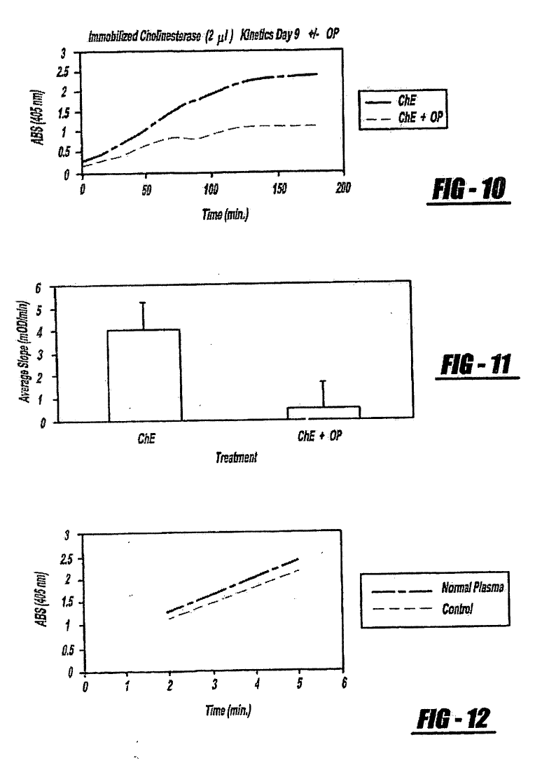

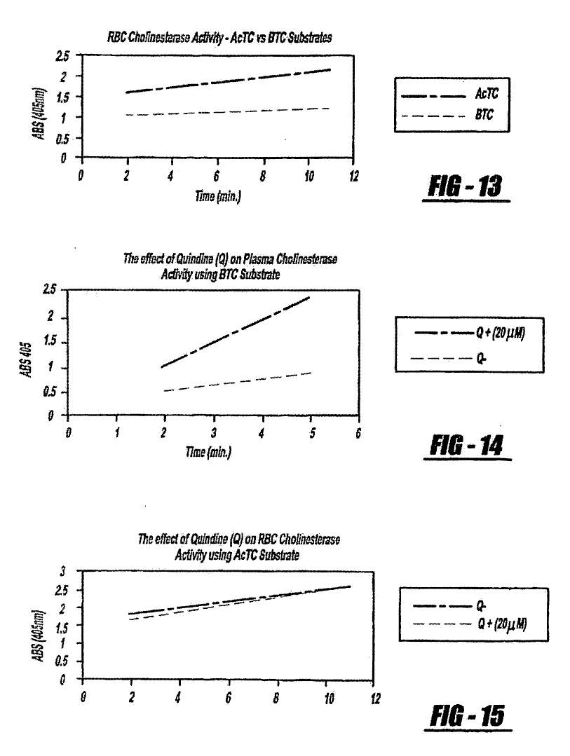

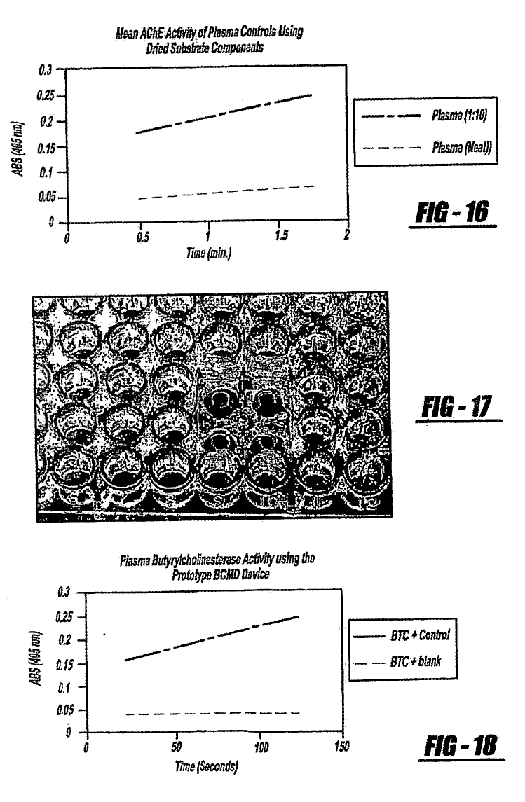

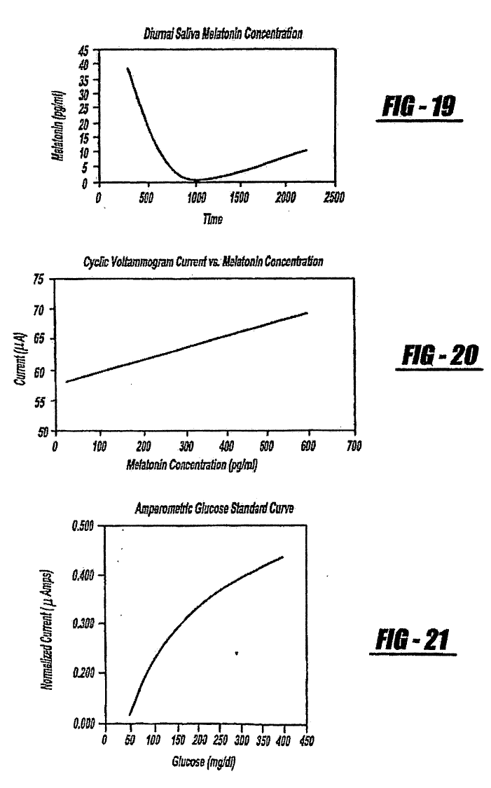

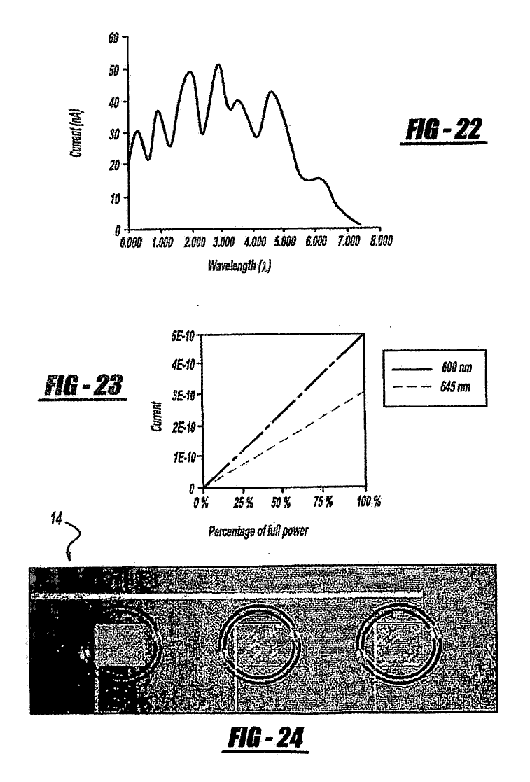

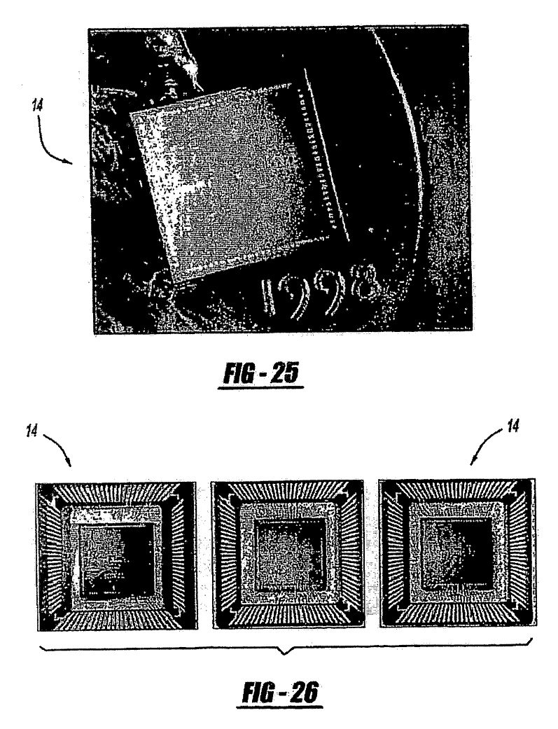

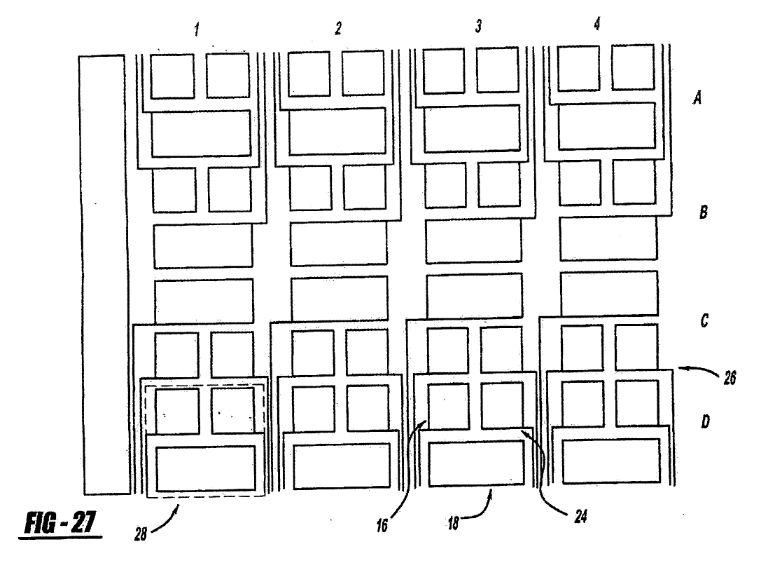

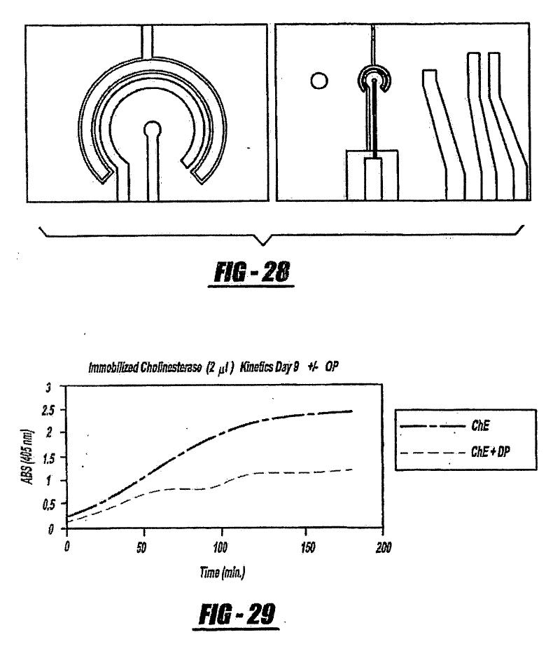

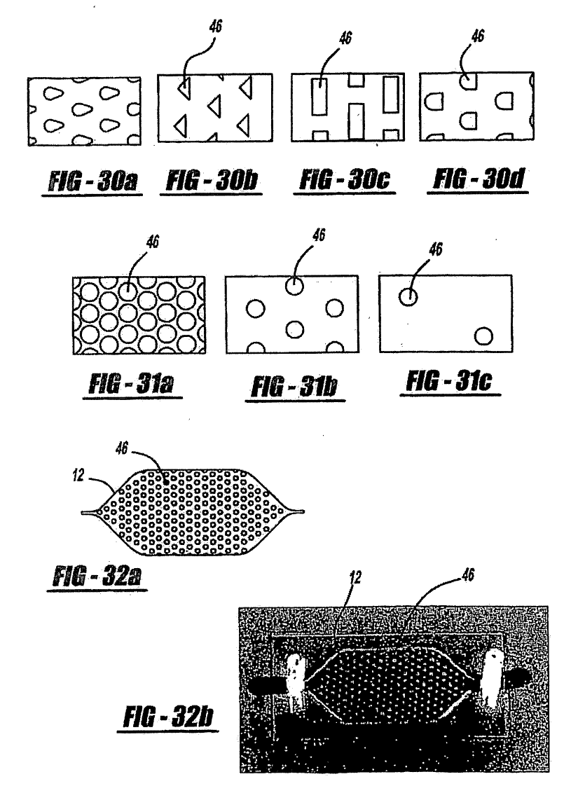

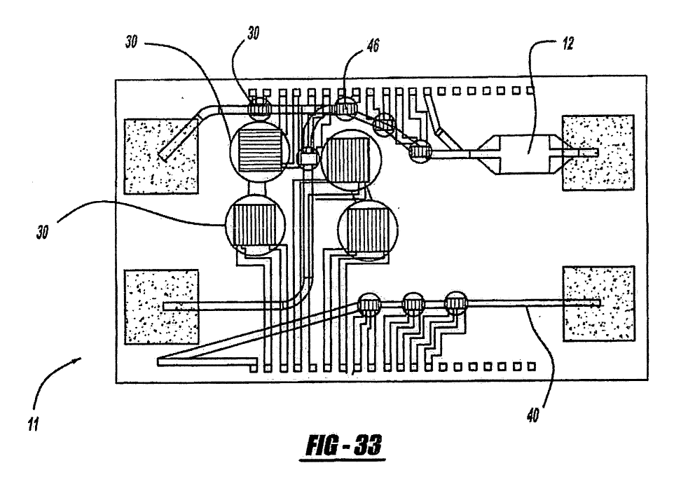

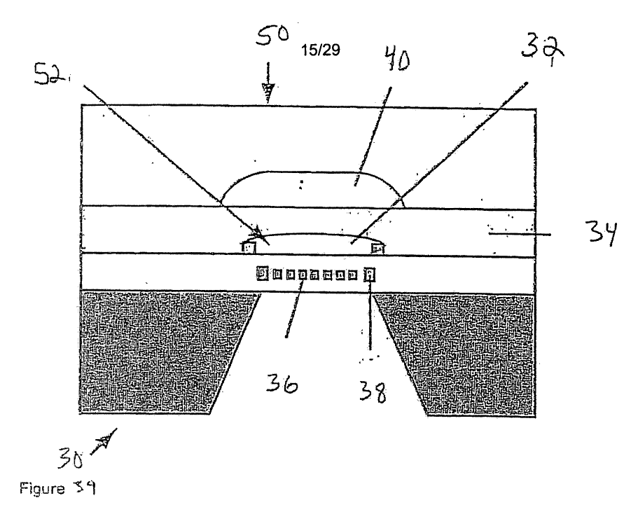

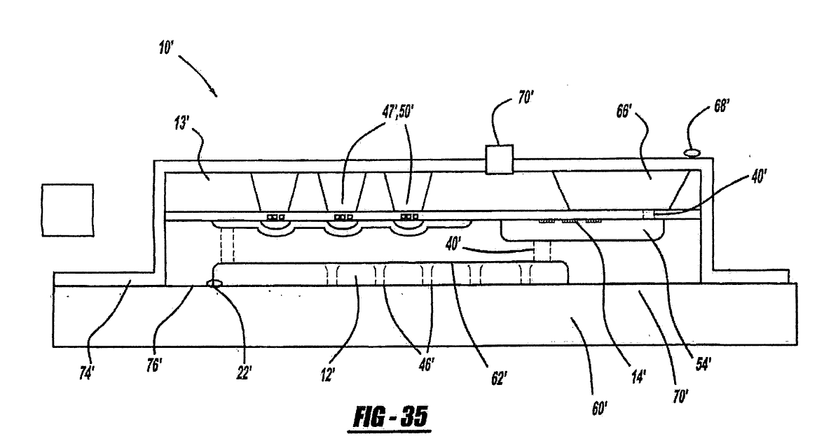

The present invention has additional advantages in that it is capable of having either a single or numerous chambers 12 (FIGS. 1 and 2). Various reactions of the fluid can take place in one chamber 12 or various other chambers 12. Movement of the fluids occurs through micro-conduits 40 connecting the chambers 12. Alternatively, reactions can take place between chambers 12 and within the micro-conduits 40 themselves. For example, a fluid can be added to a sampling chamber 12, treatment of the fluid then occurs along the micro-conduit 40, and the results are obtained at an end of micro-conduit 40 or the destination site of the fluid. Various treatments of the fluid can take place within the micro-conduit 40 such as degassing, surfactant treatment, heating, incubating, mixing with reagents, and the like that can change the state of the fluid. Additionally, various membrane-based, enzymatic, potentiometry, amperometric, electrochemical, and immunological tests can be performed within the chambers 12 or micro-conduits 40.

The agent delivery device 10 of the present invention does not require separation and/or purification of fluids before performing assaying as in typical ELISA assays. All purification and preparation steps can occur within the device of the present invention (e.g., chromatography, primary incubation with antibody, enzymatic degradation, blood cell separation, blood cell lysis, and the like). Additionally, the agent delivery device 10 of the present invention is smaller than any other system that is utilized to perform conventional ELISA based assays. The present invention utilizes and requires significantly fewer quantities of antibodies, reagents, chromophores, samples, physical space, energy, and incubation time. The microscopic nature of the agent delivery device 10 of the present invention is more amenable to temperature regulation; thus, making the assays more precise and accurate, as well as reducing incubation periods (e.g., temperature control can be performed on the device to utilize integrated polysilicon heaters and thermocouples/thermistors). The size of the agent delivery device 10 also allows multiple assays to be run on a single dipstick-type device to provide color-coded testing results more useful for the layperson via in-home testing. Thus, multiple background, standards, sample duplicates, and the like can all be performed on a 1×1 inch device, which increases accuracy through statistical analysis. Alternatively, the device can be of a smaller size such as in the micro or nano range.

As mentioned above, the agent delivery device 10 of the present invention utilizes significantly less power than conventional microfluidic devices. It is compatible with standard CMOS fabrication and therefore the controlling circuitry can be integrated onto the substrate. It is calculated that less than 700 μW of power is necessary to achieve a pumping rate of 10 μL/min and that pumping rates of 100 μL/min are achievable with this design. Pumping volumes are accurate to within 5 nL volumes.

### Feedback Control Unit

The device of the present invention can perform various assays such as an ELISA. The agent delivery device 10 of the present invention is capable of performing various tests on a single, small unit sensor system without the aid, or need, of external equipment (i.e., laboratory-on-a-chip). However, the device can be optionally linked to an external electrical source, power source, computer unit, or palm pilot as desired by the user either directly with wires or via telemetry. The device 10 of the present invention can also be constructed as an instrumentless device and can provide easily readable visual indicia of a positive and/or negative test.

When iontophoresis has been used to obtain transdermal interstitial fluid samples in the prior art devices, a troublesome tingling sensation was experienced by patients from the large area electrodes employed in the study (10 cm²). Such problems are overcome by the agent delivery device 10 of the present invention, which has a smaller area electrode (1 cm²) with an equivalent current density that does not produce as significant a “side-effect”; however, the reduced surface area results in a significantly reduced volume of drawn interstitial fluid. The device 10 of the present invention has numerous advantages over currently existing devices. For instance, the present invention is minimally invasive and measures nanoliter and microliter amounts of fluids and not milliliter amounts. By reducing the test volume required for analysis by three orders of magnitude, the surface area of the agent delivery device 10 can be significantly reduced without affecting the ability of the agent delivery device 10 to perform the necessary functions. The agent delivery device 10 is able to be so much smaller because of the microscopic semiconductor sensor arrays. The agent delivery device 10 containing a feedback control unit continuously monitors interstitial fluid in near real-time, is a small patch, approximately 10 mm×10 mm, that contains low power micro-fluidic pumps for transporting fluid samples, micro-fluidic conduits and valves for routing interstitial fluid samples and calibration solutions, platinum electrodes for electrical stimulation of the skin, microscopic semiconductor sensor arrays to detect glucose, ions, and other analytes, and electronic circuitry to control the pumps and valves as well as to provide integration with existing data-logging, telemetry, and device (pump) control systems. A schematic view of the complete micro-fluidic system, including transdermal sampling chamber and sensor array chamber, and a CAD drawing of the device is shown in the [FIG. 35]. Platinum electrodes can be integrated into the sampling chamber to facilitate iontophoretic methods to sample interstitial fluids.

Lyophilized enzyme detection chemistries can be incorporated into the device in the form of membranes on the assay pads. The membrane coated assay pads undergo calorimetric changes in response to analyte concentration. The device incorporates various microscopic, solid-state, photo diode sensors that can be plugged into a hand-held or laptop computer to objectively monitor the assay results. Alternatively, potentiometric and/or amperometric sensors can be employed. Thereby, simple assays or complex enzyme or antibody assays can be utilized.

The agent delivery device 10 of the present invention has numerous embodiments. One embodiment is directed towards a micro-electro-mechanical system (MEMS) based agent delivery device 10 including at least one sampling chamber 12. The device can optionally include micro-conduits 40, sensor arrays 14, a microfluidic system 11, and an external monitoring system 44. The agent delivery device 10 can simply include one or multiple chambers 12 (i.e., sampling, reacting, and/or sensing). If there are multiple chambers 12, then they can be in communication with each other via micro-conduits 40. Alternatively, other embodiments are directed towards a agent delivery device 10 including a sampling chamber connected to either reaction chambers 12 and/or sensor chambers 12 having sensor arrays 14. In any of the embodiments of the present invention, the agent delivery system or device 10 can be placed on an attachable means such as a patch, Band-Aid, or other disposable sensor system. The agent delivery device 10 can be placed directly onto the skin of a subject in order to obtain samples.

The chamber 12 (i.e., sampling, reacting, and/or sensing) of the present invention is generally illustrated in FIGS. 1 and 2. The chamber 12 provides for an area for placing the fluid, performing chemical reactions, sensing or detecting agents within the fluid, and/or collecting or storing the fluid. A simple one-step process can occur in one or more of the chambers 12. If numerous chambers 12 are utilized, these chambers 12 can perform required separations, measurements, and analyses of the fluid. For example, the chamber 12 can be used to lyse whole cells such as red blood cells by utilizing salts, chaotropes, heat, and any other similar reagents known to those of skill in the art. Additionally, certain chambers 12 can be utilized to contain just cells, while other chambers 12 contain only plasma therein. The actual structural components of the chambers 12 are outlined below and illustrated in the attached figures.

The chamber 12 can have various designs that have a flap or membrane covering the chamber 12 therein as well as configurations of supports 46 to act as stand-offs to prevent occlusion by the skin or to increase mixing and disrupt flow of the fluids therein. The supports 46 can vary in size and shape. For example, the bottom of the supports 46 can have a teardrop shape, oval shape, triangular shape, square, rectangular, cylindrical, and the like, while the top of the supports 46 is narrower or the same size and shape as the bottom portion thereof. The supports 46 also vary in size (i.e., volume) and shape in order to increase the volume capacity of the chamber 12.

The fluids within the agent delivery device 10 of the present invention primarily move via mechanisms including, but not limited to, capillary action, diffusion, microfluidic pumps, gravity, mechanical action, peristaltic action, pneumatic action, and any other similar mechanism known to those of skill in the art. The fluids can initially diffuse through membranes located on the device of the present invention and into various chambers 12. In other embodiments, there is no movement through a membrane.

The fluids move from chamber 12 to chamber 12 and within micro-conduits 40. Alternatively, active mechanical pressure induced by microfluidic pumps can aid in the movement of the fluids. For instance, positive or negative pressure on a membrane flap can move the fluids or active mechanical movement of micro-pumps 47′ or micro-actuators 30 can provide enough force to drive the fluids.

The micro-conduits 40 can be made of numerous materials as listed above. Additionally, the micro-conduits 40 can contain within the liner of the tube, placed in the tube or within the tube materials itself, various chemicals or reagents. The chemicals or reagents that are contained within the micro-conduits 40 or are impregnated within the micro-conduits 40 vary according to desired outcomes and reactions. For instance, the micro-conduits 40 can be coated with heparin to prevent clotting of blood, any surfactant to prevent bubbling of the fluid sample, charcoal to separate steroids, and any other similar substances known to those of skill in the art. Moreover, the micro-conduits 40 can be used to perform various treatments or reactions so that as the fluid sample travels along the micro-conduits 40, the reaction or treatment occurs and thus by the time the fluid sample reaches a designated chamber 12 or other location, the reaction or treatment is finished.

As discussed above, the agent delivery device 10 of the present invention can also include a microfluidic system 11 that aides in the quantitative and/or qualitative determination of the fluid samples. The microfluidic system 11 includes various components including, but not limited to, microfluidic pumps 47′, microfluidic devices, additional chambers 12, microfluidic valves 50, microfluidic actuators 30, DNA chips, ports, micro-conduits or tubes 40, electrodes, and deflectable membranes made of materials such as glass, plastic, rubber, and any other similar materials known to those of skill in the art. A more detailed description of the microfluidic system is set forth in PCT/US01/27340, filed Aug. 31, 2001, which is incorporated herein by reference.

The microfluidic system 11 includes microfluidic actuators 30, which are the driving mechanism behind various components of the microfluidic system 11. The micro-fluidic valves 50 have various pressures and temperatures required for their actuation. The microfluidic pump 47′ is selectively controlled and actuated through an integrated CMOS circuit or computer control, which controls actuation timing, electrical current, and heat generation/dissipation requirements for actuation.

Integration of control circuitry is important for the reduced power requirements of the present invention. Feedback provides the basis of automated adjustment of circuitry within the micro-actuator 30.

The microfluidic actuator 30 includes a closed cavity 52, flexible mechanism 34, and expanding mechanism 32. Fabrication of microfluidic actuators 30 is accomplished by generating electron-beam and/or optical masks from CAD designs of the micro-fluidic system. Then, using solid-state mass production techniques, silicon wafers are fabricated and the flexible mechanisms 34 for the microfluidic actuators 30 are subsequently placed on the chips.

In the microfluidic system 11 without integrated circuitry, the control circuitry is produced on external breadboards and/or printed circuit boards. In this manner, the circuitry is easily, quickly, and inexpensively optimized prior to miniaturization and incorporation as CMOS circuitry on-chip that can be controlled manually, or through the use of a computer with digital and analog output. Optimized CMOS circuitry, modeled utilizing CAD solid-state MEMS and CMOS design and simulation tools, is integrated into the active device making it a stand-alone functional unit.

Using an arbitrary waveform generator, and/or computer controlled digital-to-analog (d/a) and analog-to-digital (a/d) PCI computer cards (for example, the PCIM1016XH, National Instruments) the optimal operating parameters (i.e., stimulatory waveform patterns) are configured to generate peristaltic pumping action.

Electronic control of the micro-actuators 30 is optimized to maximize flow rates, maximize pressure head, and minimize power utilization and heat generation. Another parameter that is evaluated includes the temperature profile of the medium being pumped. To minimize power consumption and heat generation, a resistor-capacitor circuit is utilized to exponentially decrease the voltage of the sustained pulse. Further, integrated circuitry initiation and clocking of the circuitry provide control of the second-generation actuators.

An e-prom can also be included on-chip to provide digital compensation of resistors and capacitors to compensate for process variations and, therefore, improve the process yield. Electrical access/test pads are designed into the chips to allow for the testing of internal nodes of the circuits.

The flexible mechanism 34 deflects upon the application of pressure thereto. In one embodiment, the flexible mechanism 34 is screen-printed over the expanding mechanism 32 utilizing an automated screen-printing device, a New Long LS-15TV screen-printing system. The flexible mechanism 34 is very elastic and expands many times its initial volume as the expanding mechanism 32 under the flexible mechanism 34 is vaporized. Due to the large deflection, it is possible to completely occlude a micro-conduit 40 with this flexible mechanism 34, hence providing the functionality of an electrically actuated microfluidic valve 50. The present invention can also apply the flexible mechanism 34 with syringe or pipette devices or spin coat it on the entire wafer. Photo curable membrane can also be used to pattern the flexible mechanism 34 on the wafer.

A wide variety of commercially available polymers can be utilized as the flexible mechanism 34, including, but not limited to: Polyurethane, PVC, and silicone rubber. The actuator flexible mechanism 34 must possess elastomeric properties, and must adhere well to the silicon or other substrate surface. A material with excellent adhesion to the surface, as well as appropriate physical properties, is silicone rubber.

In an embodiment of the microfluidic system 11, the flexible mechanism 34 is made of silicone rubber. The silicone rubber can be dispensed utilizing automated dispensing equipment, or can be screen-printed directly upon the silicon wafer. Screen-printing methods have the advantage that the entire wafer, containing hundreds of pump and valve actuators, can be produced at once. By varying the amount of solvent in the polymer, such as silicone rubber, the flexible mechanism 34 thickness and its resulting physical force characteristics can be precisely controlled.

The flexible mechanism 34 can serve the dual purpose of actuation as well as serving as the bonding material used to attach the liquid flow channels to the silicon chip containing the actuators. By covering the entire area of the chip with the flexible mechanism 34, with the exception of the sensing regions and the bonding pads, the glass or plastic channels can be “glued” to the actuator containing silicon chip. This method provides additional anchoring and strength to the actuation flexible mechanism 34, and allows the actuation area to encompass the entire actuation chamber. The only drawback to this method is potential protein and/or steroid adsorption onto the micro-conduits 40. However, with proper flexible mechanism 34 selection and chemical treatment, molecular adsorption can be minimized, or a second, thin, inert layer can be used to coat the flexible mechanism 34.

The expanding mechanism 32 selectively expands the cavity defined by the flexible mechanism 34 thereof and thereby selectively flexes the flexible mechanism 34. The expanding mechanism 32 can be made of various materials. In one embodiment, the expanding mechanism 32 is a hydrogel material, which contains a large amount of water or other hydrocarbon medium, which is vaporized by the underlying heating mechanism 36. In this embodiment, the volume of hydrogel needed to produce the desired actuation and pressure for the flexible mechanism 34 is approximately 33 pL. With this design, approximately 97% of the energy generated by the heating mechanism 36 is transferred into the hydrogel for vaporization.

A practical technique for the microfluidic pumping of moderate volumes of liquid is through the use of peristaltic pumping utilizing pneumatic actuation. The integrated microfluidic system 11 of the present invention is designed to sample small amounts of interstitial fluid from the body on a continuous basis. In order to analyze the microscopic volumes, silicon micro-machining methods and recent improvements in membrane deposition technologies are utilized to produce a microscopic test chamber on the order of 50 nL in volume, roughly 3-4 orders of magnitude less volume than current systems. In addition to the improved response time, the reduction to microscopic volumes allows the use of very small amounts of calibration solution to effect calibration and rinsing, hence reducing the overall size of the package. In some systems the calibration solutions are a significant portion of the entire package (MALINKRODT MEDICAL/IL) where, even though miniature sensors are used, liters of calibration solutions are necessary.

In one embodiment, the microfluidic pump 47′ design is based upon electrically activated pneumatic actuation of a micro-screen printed silicon rubber membrane. Generally, the pump includes the microfluidic actuator 30 including a closed cavity 52, flexible mechanism 34 defining a wall of the closed cavity 52, and expanding mechanism 32 disposed within the closed cavity. The flexible mechanism 34 deflects upon the application of pressure thereto and the expanding mechanism 32 selectively expands the cavity and thus flexible mechanism 34 and thereby selectively flexes the expanding mechanism 32.

The microfluidic actuator 30 is based upon electrically activated pneumatic actuation of a micro-screen-printed or casted flexible mechanism 34. The peristaltic pump generally includes three microfluidic actuators 30 placed in series wherein each microfluidic actuator 30 creates a pulse once it is activated. By working in tandem, the microfluidic actuators 30 peristaltically pump fluids. The optimal firing order and timing for each microfluidic actuator 30 depends upon the requirements for the microfluidic system 11 and are under digital control to create the peristaltic pumping action. The advantage of pneumatic actuation is that large deflections can be achieved for the flexible mechanism 34. To actuate the flexible mechanism 34, a vaporizable fluid is heated and converted into vapor to provide the driving force.

Utilizing an integrated heating mechanism 36, the expanding mechanism 32 is vaporized under the flexible mechanism 34 to provide the pneumatic actuation. This actuation occurs without the requirement of utilizing external pressurized gas.

The liquid or gaseous fluid being pumped serves the purpose of acting as a heat sink to condense the vapor back to liquid and hence return the flexible mechanism 34 to its relaxed state when the integrated heating mechanism 36 is inactivated. A temperature sensor 38 is integrated adjacent to the actuator to monitor the temperature of the microfluidic integrated heating mechanism 36 and hence, expanding mechanism 32.

Once the integrated heating mechanism 36 is activated, vaporization of the expanding mechanism 32 takes place. The expanding mechanism 32 component imposes a pressure upon the flexible mechanism 34 causing it to expand and be displaced above the integrated heating mechanism 36 and reduces the volume of the chamber. This methodology can be utilized to displace fluid between the flexible mechanism 34 and the walls of the chamber (pumping action), to occlude fluid flow through the chamber (valving action), to provide direct contact to the glass substrate to effect heat transfer, or to provide the driving force for locomotion of a physical device (i.e., as in a walking caterpillar and/or a swimming paramecium with a flapping flagella, in which case the glass chamber encompassing the micro-actuator 30 is not used).

In one embodiment, the temperature of the saturated liquid hydrogel, at 1 ATM, is assumed to be 100° C. The heat flux to the air, through the back of the integrated heating mechanism 36, is calculated to be 1263 W/K-m². The total heat flux through the device is calculated to be 46,995 W/K-m² with a total flux from the heating mechanism 36 of 47,218 W/K-m² (i.e. 97% efficiency of focused heat transfer). In this embodiment, the temperature of the inactive state hydrogel varies between 86° C. and 94° C.

The temperature of the activated, vapor state hydrogel is approximately 120° C., which is the saturation temperature for steam at 2 ATM. The heat transfer coefficient for convection can be calculated directly from the thermal conductivity.

The heat flux to the air through the back of the integrated heating mechanism 36 is 2818 W/K-m². The heat flux through the device is 21,352 W/K-m² with a total flux from the integrated heating mechanism 36 of 24,170 W/K-m². When the aqueous component of the hydrogel is completely in the vapor state, there is no fluid in the channel and the thin film of solution between the flexible mechanism 34 and the glass is approximately at 60° C. These values and calculations vary according to the type of actuator, valve, pump, and micro device being used.

In an embodiment of the present invention, the volume of the expanding mechanism 32, in this case, liquid hydrogel, is determined based on the volume of vapor needed to expand the flexible mechanism 34 completely at 2 ATM using the ideal gas law. This assumption is valid because the temperatures and pressures are moderate. The volume of liquid hydrogel necessary to achieve this volume of gas at this pressure, assuming the hydrogel is 10% water and all of the water is completely evaporated, is 0.033 nL. Cylindrically shaped sections of hydrogel are utilized within the microfluidic actuator 30. This shape has been chosen to optimize encapsulation by the flexible mechanism 34. The cylinders have either a diameter of approximately 140 μm and a height of 2.14 μm, or a diameter of 280 μm with a height of 0.54 μm (identical volumes, different orientation to the heating element). Of course, the shapes and volumes vary according to the type of expanding mechanism 32 being used. For example, photocurable liquid hydrogels have different parameters.

The integrated heating mechanism 36 is poly-silicon, but can be any similar material or mechanism, such as direct metals, known to those of skill in the art. Because of its high thermal conductivity, the silicon substrate acts as a heat sink. To reduce thermal conduction to the silicon substrate, a window in the silicon, located beneath the integrated heating mechanism 36, provides the expanding mechanism 32 with an isolated platform. This window is only slightly larger than the integrated heating mechanism 36 to maintain some thermal conduction to the substrate. After the microfluidic actuator 30 is energized, thermal conduction to the silicon provides decreased time to condense the liquid in the expanding mechanism. This decreases constriction time and provides improved pumping rates. If the window is significantly larger than the microfluidic actuator 30, there is no heat conduction path to the substrate, hence increasing condensation time and decreasing the maximal flow rate.

A polymeric hydrogel (or hydrocarbon) can be utilized to provide a physically supportive structure that withstands the application of flexible mechanism 34 as well as to provide the aqueous component required for actuation. Several commercially available materials meet these requirements. A hydrogel is selected that contains approximately 30% aqueous component that vaporizes near 100° C. Several materials have been identified, each of which is suitable in this application, including, but not limited to, hydroxyethylmethacrylate (HEMA) and polyvinylpyrrolidone (PVP).

Additionally, hydrocarbons can be used since they possess lower boiling points than aqueous hydrogels, and therefore require less power to effect pneumatic actuation. Dispensing hydrogel (or hydrocarbon) into the desired location is accomplished utilizing one of three methods. First, a promising method for patterning the hydrogel is to utilize a photopatternable-crosslinking hydrogel. The hydrogel is cross-linked by incorporating an UV photo-initiator polymerizing agent within the hydrogel that cross-links when exposed to UV radiation. Using this technique, the hydrogel is evenly spun on the entire wafer using standard semiconductor processing techniques. A photographic mask is then placed over the wafer, followed by exposure to UV light. After the cross-linking reaction is completed, excess (non-cross-linked hydrogel) is washed from the surface.

The second method involves dispensing liquid hydrogel into well rings created around the poly-silicon integrated heating mechanism 36. These wells have the ability to retain a liquid in a highly controlled manner. Two photopatternable polymers have been utilized to create microscopic well-ring structures, SU-8 and a photopatternable polyimide. These well rings can be produced in any height from 2 μm to 50 μm, sufficient to contain the liquid hydrogel. Once the hydrogel solidifies, flexible mechanisms 34 can be deposited over them. This can be accomplished in an automated manner utilizing commercially available dispensing equipment.

In a third alternate method, a pre-solidified hydrogel is used that has been cut into the desire size and shape. This is facilitated by extruding the hydrogel in the desired radius and slicing it with a microtome to the desired height, or by spinning the hydrogel to the desired thickness and cutting it into cylinders of the desired radius. Utilizing micromanipulators, the patterned gel is placed in the desired area. This process can also be automated.

It is assumed that the temperature on both sides of the SiO₂ that encapsulates the integrated heating mechanism 36 is constant, and that heat flux in each direction is dependent upon the heating mechanism 36 temperature and both sides are resistant to heat flow either through the device or to an air pocket on the integrated heating mechanism 36 backside. Steady-state heat flow through the entire actuator, for the fully actuated state, the intermediate state, and the resting state are modeled. These data are calculated for the static case during which time no fluid flow is occurring (i.e. steady-state; the system is poised at 100° C., waiting to be initiated). The fluid temperature is greater for the contracted state since the liquid hydrogel conducts heat at a greater rate than vapor. Once fluid flow is initiated, the temperature of the solution is raised by only a few degrees Celsius.

A typical problem experienced with many microfluidic designs revolves around the methodology for mixing of solutions and reagents. The microfluidic peristaltic pump 47′ design of the present invention provides mixing action in concert with the pumping action. To construct the microfluidic valves 50 and pumps 47′ in a manner compatible with the sensor technologies and to integrate the entire system on a single silicon chip, the pump is preferably fabricated using planar MEMS technologies that do not require special wafer bonding, although other methods of fabrication can also be used as are known to those of skill in the art.

For encapsulating a liquid within a silicone rubber membrane, micro-machining techniques, including wafer bonding of multiple chips, are used by others to create a cavity where the liquid is stored. This requires several machining steps to produce the actuator, reducing the overall yield of functional pumps and valves, and increasing the cost.

By properly placing the planar actuators within the fluidic channels, micro-pumps, fluidic multiplexers, and valves can be formed. CAD/CAM tools are used to design the photo-masks. This can be accomplished in conjunction with the design of the fluidic channels, ports, and test chambers.

The pneumatically actuated membrane is utilized to produce the microfluidic valves. The microfluidic actuator's silicone rubber membrane is very elastic and expands many times its initial volume as the liquid under the membrane is vaporized.

At least two techniques for the valving of solutions can be used. The first utilizes the flexible mechanism 34 actuation to completely fill a microfluidic channel when actuated, hence providing the functionality of an electrically actuated microscopic valve. The second utilizes the flexible mechanism 34 to occlude an orifice to block fluid flow.

The pneumatically actuated membrane is also utilized to produce the microfluidic pumps. The microfluidic actuator's flexible membrane 34 is very elastic and expands many times its initial volume as the liquid under the membrane is vaporized. The microconduits 40 are designed such that all media flow is in the laminar regime while minimizing fluid volume, dead volume, and residence time.

Further, the routing of the microconduits 40 is designed such that the required calibration and wash solutions can be routed into the sensing chamber 12. The conduits 40 and sensing chamber 12 accommodate approximately 50 nL volumes of solution.

Once modeled and optimized, photomasks are created for the fluidic system. Valves at the various ports are optimally designed to start and stop the flow of the various calibration and wash solutions.

In one embodiment, the integration of a sampling system or microfluidic system 11 to the device 10 allows transdermal-sampling techniques for the acquisition of interstitial fluids. This sensing chamber 12 has a maximized surface area within the confines of the device 10 and an extremely minute volume to reduce the required sample volume and to decrease the sampling time. This sensing chamber 12 is micro-machined into the backside of the glass fluidic channel chip.

For mobile applications, automated control of the pumps, valves, and sensors is required to continuously monitor and calibrate the microscopic “lab-on-a-chip” devices. Using integrated electronics, the sensors 14 can be calibrated on a regular basis in an automated manor that is transparent to the user, ensuring accuracy of the data obtained. The sensing system also requires integrated circuitry to buffer the signals, reduce noise, transduce the chemical concentrations into electronic signals, and analyze the signals, allowing untrained personnel to utilize the device.

Another application for integrated circuitry is for the telemetric communication of the device with a base unit, which can then relay the information to a remote location. Moreover, the circuitry can perform closed-loop feedback control for biological applications. For example, closed-loop feedback control can be used to inject insulin into an individual when the transdermal sensor system detects hyperglycemic levels of glucose in the transdermally sampled interstitial fluid, thereby maintaining euglycemia.

The sensors 14 are fabricated in a three-mask process with two metal layers, silver and platinum. Since these metals are difficult to etch using wet chemistry, a resist lift-off process was used to pattern them. This provided an additional advantage in allowing the use of layered materials in a metal structure to modify electrode properties and still allowed for patterning to occur in one step.

Additionally, other sensor 14 conformations can be produced in accordance with the present invention, each with differing transduction, and membrane encapsulation properties. These designs incorporate rectangular, circular, and concentric circle shaped electrodes.

In any embodiment, the microfluidic valves 50 of the present invention utilize an actuating mechanism to occlude a micro-conduit 40 and thereby decreasing or preventing fluid flow. The ability to occlude is selective, in that the valve can effectively open and close a passageway of the micro-conduit 40. The microfluidic actuators 30 are the driving mechanism behind the microfluidic valves 50 of the present invention.

For a mono-stable microfluidic valve 50, it is assumed that the temperature on both sides of the Si0₂ that encapsulates the integrated heating mechanism 36 is constant, and that heat flux in each direction is dependent upon the integrated heating mechanism 36 temperature and the general resistance to heat flows either through the device or to the air from the backside. In order to isolate the heater, a cavity is etched in the backside of the wafer, providing thermal isolation. The microfluidic valve 50 requires continuous power to maintain a closed-stated position. Utilizing the integrated heating mechanism 36, an expanding mechanism 32 is vaporized under the flexible mechanism 34 thereby providing the pneumatic driving force required for expanding the flexible mechanism 34 and hence occluding the micro-conduit 40. The mono-stable, normally open microfluidic valve 50 utilizes a single actuator to effectively actuate the valve. As the hydrogel is expanded, the silicone rubber of the actuator completely occludes the micro-conduit 40 to effect valving of the solution. While the normally open microfluidic valve 50 is less complicated to construct, it requires continuous power or pulsed power to keep the valve closed.

A bi-stable microfluidic valve 50 is also capable of being utilized. The bi-stable valve 50 is designed that utilizes lower power consumption and a wax material to provide passively open and passively closed functionality, i.e. bi-stability. Thus, power is only required to transition from one state to the other. The bi-stable valve design is based upon the utilization of a moderate melting point solid, such as paraffin wax, which possesses a melting point between 50° C. and 70° C.

The bi-stable microfluidic valve 50 similarly utilizes actuating mechanisms to occlude the micro-conduit 40. The mono-stable microfluidic valve 50 can only provide the functionality of a normally open valve. During the period that the valve must be maintained in a closed position, continuous power must be applied. The bi-stable microfluidic valve 50 utilizes microfluidic actuators 30 to provide both zero-power open and closed functionality.

The bi-stable microfluidic valve 50 utilizes a total of three microfluidic actuating mechanisms 30. Any number of actuating mechanisms 30 can be used without departing from the spirit of the present invention. Two actuating mechanisms are physically connected by a micro-conduit 40 formed under the membrane and are filled with a low melting point solid such as paraffin wax as opposed to an aqueous hydrogel (see above for mono-stable actuation). The third is a standard design micro-actuator filled with an aqueous hydrogel connected by the expansion chamber to the middle wax filled actuator. The first two microfluidic actuators 30 are activated causing the wax to melt. The third, standard, microfluidic actuator 30 is then activated, providing pneumatic force on the wax containing actuators, causing the orifice containing chamber to close. The wax is then allowed to solidify. Again, the advantage of this valve is that it requires power only to transform from the stable open to the stable closed state.

In the open state, medium in the channel readily flows. To switch from the open state to the closed state, the wax is melted and the pneumatic microfluidic actuator 30 on the right is expanded. This creates pressure outside the middle actuator, forcing the paraffin into the smaller left chamber, expanding the membrane, thereby blocking fluid flow. The wax is allowed to solidify, after which the power can be removed from the actuator providing the driving force pressure, resulting in an electrically passive closed state. To transition from the closed state to the open state, the wax is melted and membrane tension forces the wax from the small left chamber back into the middle chamber. The micro-valve design provides bi-stable functionality, which only requires power to switch between each state, but is completely passive once in either the open or closed position.

The use of polydimethylsiloxane (PDMS) in multiple layers to directly produce the three-dimensional structures of the microfluidic system is a technique well suited to mass production. This technique has the advantages of allowing an entire wafer of chips to be packaged simultaneously and of being compatible with integrated circuitry. This process is fairly complex, requiring multiple photo patterning of the devices and the application of a top layer to complete the structure. Despite the manufacturing challenges, this method is capable of creating three-dimensional microfluidic systems. PDMS has the following properties: low glass transition temperature, low surface energy, high permeability of gases good insulating properties, and very good thermal stability. The properties of PDMS can be altered such as to convert the surface from hydrophobic to hydrophilic. This can be accomplished by numerous methods known to those of skill in the art including, but not limited to, oxygen plasma treatment, hot acid treatment, surface coating with polyurethane, and surfactant treatment.

The sensors 14 of the present invention include at least one amperometric sensor, and at least one potentiometric sensor. The sensors of the present invention can detect neuronal action potentials and the resulting release of neurotransmitting and/or hormones. The sensors can also detect the diffusion, dispersion, degradation, and re-uptake of neurotransmitters, hormones AND/OR other cellular metabolites. Examples of such sensors 14 are known to those of skill in the art and more specifically, sensors are disclosed in co-pending U.S. patent application Ser. No. 10/111,964, filed May 2, 2002.

Coulometry is the determination of charge passed or projected to pass during complete or nearly complete electrolysis of an analyte, either directly on the electrode or through one or more electron transfer agents. The current, and therefore analyte concentration, is determined by measurement of charge passed during partial or nearly complete electrolysis of the analyte or, more often, by multiple measurements during the electrolysis of a decaying current and elapsed time. Once the hydration shell has been established around the electrode, the decaying current results from the decline in the local concentration of the electrolyzed species caused by the electrolysis. A compound is immobilized on a surface 26 when it is physically entrapped on or chemically bound to the surface.

Electrochemical detection, specifically amperometry, has been used in the past in relatively unsophisticated applications, for example, detecting and quantifying eluted molecules at the end of chromatographic columns (Kissinger et al, 1984).

The main limitations of amperometry are its low specificity and sensitivity. The present invention takes advantage of this technique's speed and overcomes its limited specificity and sensitivity. First, to enable the amperometric sensors to detect multiple neurotransmitters independently, the sensors employ two particular forms of amperometry; cyclic and constant voltage voltammetry. Second, utilizing a micro-screen printing device, such as a New Long LS-15TV, several different selectivity membranes can be applied over the individual sensors to eliminate background measurement of unwanted compounds (such as ascorbic acid) and impart specificity onto the microscopic electrodes including the sensor (Goldberg et al, 1994). Finally, by encapsulating the sensors 14 leads with silicon nitride, which is a substrate that neurons can be made to readily attach, the sensor array is in very close apposition to the secreting neurons allowing measurement of the relatively high neurotransmitter concentrations in the immediate vicinity of the axon, prior to degradation, dilution, dispersion, and re-uptake.

An amperometric process, cyclic voltammetry, is a technique whereby a cyclically repeated triangular waveform of potential is applied between the working and counter electrodes. Individual analytes, such as neurotransmitters, have characteristic oxidation and reduction potentials based on their chemical moieties (Adams, 1969; Dryhurst et al, 1982). When the voltage between the electrodes reaches the oxidation potential of a particular neurotransmitter that molecule oxidizes. Oxidation is a process whereby an electron is stripped from the molecule. The counter electrode absorbs the oxidatively produced electrons, effectively transducing chemistry into electricity. The flow of electrons per unit of time is current, which is proportional to the number of molecules being oxidized. The voltage at which this oxidatively produced current is obtained provides information useful for identifying the analyte such as neurotransmitter, hormone or cellular metabolite being measured (Dryhurst et al, 1982; Baizer et al, 1973).

Other embodiments of the sensor array can include, but is not limited to, additional components such as various separating and purifying mechanisms, heating elements to aid in the lysis of cells, adding and mixing mechanisms, and degassing mechanisms to remove air bubbles. Moreover, various agents can be added to the present invention including, but not limited to, surfactants, primary antibodies to start ELISA reactions, other enzymes to start desired reactions, color reporters (HRP), luminescent agents, or other indicators, and any other chemicals or substances known to those of skill in the art.

The membrane interface chamber 12′ can be removably attached to the body portion 13′ so that it can be disposed of, for sterility issues, and making the testing chamber 54′ reusable. As shown in FIG. 37, the membrane interface chamber 12′ can be removably secured to the body portion 13′ through the use of a die-locker 78′ for locking the membrane interface chamber 12′ in place and a spring 80′ for releasing the membrane interface chamber 12′. Any other suitable lock and release mechanism can also be used.

The testing chamber 54′, having the properties of the sensing chamber 12 including various sensors (such as a sensor array 14′), is a housing in which a reaction(s) is performed on the captured interstitial fluid. The testing chamber 54′ is operatively connected to the membrane interface chamber 12′ through at least one micro-conduit 40′. The captured interstitial fluid in the membrane interface chamber 12′ can be drawn through the micro-conduits 40′ into the testing chamber 54′ so that a reaction can be performed to determine the presence of molecules. Such reactions can be ELISA assays, or chromatography as described above, a PCR assay, an absorbance assay, a calorimetric assay, a solid-phase immunoassay, an enzyme immunoassay, a fluorescent immunoassay, or any other suitable reaction or assay.

The sensors/sensor array 14′ include at least one potentiometric and one amperometric sensor as described above. The sensor/sensor array 14′ can be covered by an array membrane as described above for the purpose of potentiometric transduction or to provide selected access by certain molecules to the sensor. The sensor/sensor array 14′ is manufactured and made of materials as described above.

The testing chamber 54′ further includes an evaporative waste disposal chamber 66′ as shown in FIGS. 35 and 36. The evaporative waste disposal chamber 66′ allows fluids from the testing chamber 54′ to be removed from the device 10′ through evaporation once a reaction has been performed. The evaporative waste disposal chamber 66′ can be operatively connected to the testing chamber 54′ by micro-conduits 40′, and can be manufactured in the same manner and with materials described above for the chambers 12.

The testing chamber 54′ can further include a signal transmitter 68′ for sending a signal either by telemetry to a microprocessor and/or to a second device for dispensing a molecule, or by electronic connection to another site on the device 10′. The signal transmitter 68′ can be any suitable signal transmitter 68′ and can be operative integrated in the agent delivery device 10′ at any suitable location. The signal can be used to report the results of the reaction(s) in the testing chamber 54′ and can be displayed to a user, either on the agent delivery device 10′ itself or on a separate microprocessing device. The signal can have a unique encoding so as to distinguish from other signals coming from other devices. The signal transmitter 68′ can operate in any suitable band such as but not limited to the wireless medical telemetry services (WMTS) band, radio frequency, or other similar frequencies capable of operating the device of the present invention. Any suitable signal transmitter 68′ can be used. For example, Bluetooth™ technology can be utilized. The telemetric signal can come from a remote device such as from a handheld control, or from a main station such as a nurse's station or any other base for monitoring people.

### Software and Computer Interface

The present invention provides a transdermal monitor with wireless technology for the purpose of transmitting data from the device to a remote computer. There are a wide variety of alternative wireless technologies that could be employed, a preferred embodiment would utilize Bluetooth™ wireless technology. Bluetooth™ was chosen for a number of reasons.

Bluetooth™ wireless technology is specifically designed for short-range (nominally 10 meters) communications; one result of this design is very low power consumption, making the technology well suited for use with small, portable personal devices that typically are powered by batteries. A typical Bluetooth™ device draws less than 0.3 mA in standby mode and an average of 5 mA in raw data mode. Bluetooth™ was designed to be simple to implement, have low power consumption and be relatively inexpensive. There is no need for a line of sight between the Bluetooth™ transponder and receiver since Bluetooth™ uses a radio link for communications. These characteristics make Bluetooth™ well suited for use in medical applications such as physician tools, diagnostic instruments and telemedicine.

A Bluetooth™ module consists primarily of three functional blocks, an analog 2.4 GHz Bluetooth™ RF transceiver unit, a baseband link controller unit, and a support unit for link management and host controller interface (HCI) functions. Bluetooth™ uses Frequency Hopping Spread Spectrum (FHSS) technology (1600 hops/second) to increase the reliability of the communication channel. The signal hops among 79 frequencies at 1 MHz intervals to give a high degree of interference immunity. Bluetooth™ devices form networks called Personal Area Networks (PANs) or piconets. Up to seven simultaneous connections can be established and maintained in a piconet. The device that establishes and controls the piconet is called a master and all other seven devices in the piconet are called slaves. These piconets are established dynamically and automatically as Bluetooth™ devices enter and leave the radio proximity. This allows many different devices to be used by many different users in a dynamic environment. Each piconet uses a slow hopping frequency with a pattern determined by the master. A master also does the timing of the network with the slaves synchronizing to the master's clock. Using this methodology, Bluetooth™ devices are capable of 723.2 kbps, which is more than sufficient for the proposed glucose monitor.

Bluetooth™ technology can be either built into an electronic monitoring device or used as an adaptor that plugs into these devices. The Bluetooth™ device contains a circuit board, power supply, Bluetooth™ core chip, Bluetooth™ RF (radio) module, interface (USB or RS232), PCM chip and audio interface for audio interface and connector for external antenna.

There are three ways of implementing Bluetooth™ wireless technology into an end product. The first is by using a Bluetooth™ module. Although it is a very expensive and inflexible method, this is the easiest method that offers fastest time-to-market solutions. The second method is to use a pre-qualified Bluetooth™ chipset. The off-the-shelf items are available in the market for integration into the system level of the product. The third method is to directly incorporate Bluetooth™ circuitry directly into the product being developed. The IP for directly incorporating Bluetooth™ into a product can be purchased from providers such as Newlogic, Ericsson or ParthusCeva.

Develop the software architecture: There are three aspects of the software system that are required: the overall software architecture, the interface between the patch and the remote computer, and the user interface. Object oriented methods were used for developing all aspects of the device's software.

The software architecture describes the relationship of the system's data objects with other data objects and with external systems. The system has two data producers: the patch and the user input, and one data consumers: the local display of data. Since each of these data objects can act relatively independently, the number and complexity of the interactions between the system's data objects are likely to be minimal.

To be able to operate within this type of environment, one must either employ a common interface or employ an interface that works with a defined subset of external systems.

SQL Server CE is a compact database for rapidly developing applications that extends enterprise data management capabilities to mobile devices. SQL Server CE makes it easy to develop mobile applications by supporting the industry-standard Structured Query Language (SQL) syntax. SQL Server CE also provides a range of data types and supports 128-bit encryption on the device for database file security.

The SQL Server CE engine exposes a broad set of relational database features while maintaining a compact footprint that enables applications using this engine to be deployed to a wide variety of PocketPC devices. The programming and operational model, which is consistent with the rest of the SQL Server family, facilitates the development of new applications and integration with existing systems. SQL Server CE is easily integrated with the Microsoft .NET Compact Framework by means of Microsoft Visual Studio .NET, thereby simplifying database application development. This allows mobile application developers to build highly extensible applications with offline data management capability for disconnected scenarios.

This is a key feature not present in existing mobile databases. SQL Server CE is particularly well suited for mobile and wireless environments as it has methods for remote data access and ensuring merge replication with SQL Server databases. Remote data access exposes data in SQL Server databases through remote execution of Transact-SQL statements and providing the ability to pull record sets to the client device for updating. SQL Server CE provides the ability to synchronize through merge replication. These data access technologies take advantage of Internet standards, including HTTP Secure Sockets Layer (SSL) encryption, through integration with Internet Information Services (IIS). This approach ensures data can be accessed reliably and flexibly, even through firewalls. These are important capabilities as MS SQL server is one of the three most commonly deployed databases and IIS is one of the two most commonly deployed web servers.

Later versions of the software can employ the Extensible Markup Language (XML) for data interactions with external systems. XML is a markup language for documents containing structured information. Structured information contains both content and an indication of the role that content plays. A markup language is a mechanism to identify structures in a document. The XML specification defines a standard way to add markup to documents. XML is an international standard and most all modern computers provide the ability to create and parse XML documents. By employing an XML-based interface, all computers are able to interact with the data provided by the PDA.

Although the shift to XML might seem like a radical departure from the SQL method described previously, it is actually an enhancement to the proposed system, not a replacement for it. This is due to the fact that both SQL Server CE and Microsoft Visual Studio .NET, the development environment of choice for mobile SQL Server CE applications, provide extensive support for building and deploying web-based XML applications. The integration of this capability can provide the broadest possible base of support for the system.

Design the user interface: Possibly the most important aspect of the software design is the graphical user interface (GUI). Aspects of this task include defining the users' interaction with the system, defining the means for inputting data into the system, and defining the data presented to the user and the format in which it is presented.

The patient has several modes of interaction with the device. Representative interactions include, but are not limited to, inputting relevant therapeutic information into the system, recalling historical data for analysis and study, and uploading data to a centralized system.

As shown above, several of the physician's interactions with the device include the entering of data. Since the core of the device is a PDA, the most obvious choice of methods for inputting this data is via on-screen buttons and/or written notes. A suite of buttons and/or free-form text fields was carefully designed to provide the physician with the greatest possible degree of flexibility while minimizing the effort to input the data. Additionally, the device can use voice input. At the least, voice input could be used by the physician to store examination notes. With the use of voice recognition, it is possible to eliminate the need for manual data input.

The GUI can be developed in such a manner as to make the device as easy to use as possible. This means that each screen has a single purpose, such as data entry, viewing results, etc., and that the most obvious controls can sequence through the screens in a typical fashion. To provide the physician with full control, all system functions can be available (probably through a menu system), though the ones that are infrequently used can require one or two levels of menu navigation to reach.

The device was developed using a MS CE.NET compliant PocketPC. The two primary reasons for choosing this platform are the wide availability of such devices with CF ports and the ease of graphically developing GUI's using Microsoft Visual Studio .NET. By using a graphical design paradigm, the software developer can more easily develop systems that are ergonomically sound and visually pleasing.

Develop a stand-alone version of the software: The distinguishing feature of the proposed system is its use of an industry standard, relational database as the core of the software aspect of the product. This contrasts with all other PDA-based programs for managing diabetes that employ proprietary, flat-file systems. Since the database is the core of the software program, the first step in developing the application is developing the database schema.

A schema is the logical structure of the database, i.e., it defines the relationships between each of the data objects contained within the database. The figure shows a preliminary sketch of a schema for this project:

The schema focuses solely on the dietary logbook aspect of the project. Additional tables for storing personal information, sensor readings, and other user supplied data can be added to this schema when development commences. There are several noteworthy features of this schema: 1. Data items are never removed from the database, instead, they are marked as being inactive. This guarantees that data analyses performed in the future can always return valid data; 2. The grouping of food items into groups greatly facilitates searching for items. This is supported by the use of many-to-many relationships that helps ensure data normalization; and 3. Since the data is being stored in a relational database, searches can be performed using any combination of criteria, thereby making it possible to quickly locate data items of interest.

The next aspect of the software development is the implementation of the GUI (see Task 5). To facilitate development, the program was developed using Microsoft Visual Studio .NET 2003, which has built-in support for PocketPC development. The tools provided permit developing applications for PocketPC's in the exact same manner as for desktop systems. Visual Studio also facilitates the development of database applications through the use of SQL-specific data objects and methods.

To facilitate usage of this system, it was necessary to populate the database, especially the Food and Group tables, with typical foodstuffs so users can immediately start entering there consumption data without first having to populate these tables. To perform this subtask, a database was identified with the necessary information that is in the public domain and can import the data into the database.

In another embodiment of the present invention, the device can be used in conjunction with a hand-held reader for electronically timing the reaction rates and provide digital read-out to automate the measurement process so as to eliminate the need for trained personnel. In this embodiment, the device includes a disposable cartridge containing the enzyme chemistry reagents, detection chambers, and microconduits, a reader containing the sensors, actuators and controlling electronics, and a hand-held read-out system.

The hand-held read out system is usable by both the clinician as well as the patient themselves. It can be designed and developed for use with the device of the present invention. The readout device can be designed as a “hand-held” readout and controlling instrument (RCI) utilizing commercially available Palm or Windows CE hand-held computers. The RCI can be utilized to provide an ergonomic display of sensor and calibration data as well as to monitor trends in the patient. The RCI can control the actuator timing to obtain more or less frequent samples and/or calibrations in a given time period. The RCI unit is also responsible for sensor data conversion utilizing the calibration parameters.

On the chip-based sensor unit, the data is stored in a digital manner until it is ready to be read by the RCI. The RCI accepts a stream of data from the sensor unit and display it in one of two different configurations. The first software implementation in the RCI is for the patient that can display subjective data. In other words, if concentrations are in a high, normal, or low range, then trend analysis providing simple exposed/not-exposed information to the patient. The second version can be utilized by the clinician or trained personnel, who can receive a readout that displays quantitative data from the sensor array and allows data output for use in any standard database or graphing program. In addition, the RCI allows the clinician to control—the acquisition device, including sampling frequency, calibration frequency, alarm settings, etc. Numerical concentration levels and trends can be displayed on a hand-held computer or PDA. Furthermore, compatible integration into a Medical database for the individual can take place.

### Device Materials and Manufacturing

The agent delivery device of the present invention can be composed of numerous materials including, but not limited to, plastic, silicone, glass, metals, alloys, rubber, combinations thereof, or any other similar material known to those of skill in the art.

Typically, the device of the present invention is manufactured by chemical etching methods known to those of skill in the art. Thus, the chambers and micro-conduits of the present invention can be etched into a base material of silicon or glass. The chambers are made out of material that is sandwiched between pieces of silicon, glass or membranes. Further, utilizing glues and other securing methods and materials known to those of skill in the art can be utilized to make the present invention. Fabrication of the microfluidic system components is based upon the development of a process flow. The fabrication process utilizes bulk silicon micro-machining techniques to produce the isolation windows, and thick film screen-printing techniques, spin coating, mass dispensing, or mechanical dispensing of actuation membranes.

Alternatively, the chambers and conduits can be produced from plastic by injection molding, micro-milling, or soft lithography. The materials of the present invention can be modified or altered according to the specific design required. Moreover, the device of the present invention can vary in size, shape, and configuration without departing from the spirit of the present invention.

### DEFINITIONS

Like structure among the several defined embodiments are indicated by primed numbers.

The terms “chamber 12,” “testing chamber 54” “sampling chamber 12,” “reacting chamber 12,” and “sensor chamber 12” are defined as an enclosed reservoir wherein fluids are retained.

The term “agent” is defined as a traceable biological or chemical component. As used herein, an “agent” is meant to include, but is not limited to environmental agents, blood markers, antigens, pesticides, drugs, chemicals, toxins, PCBS, PBBS, lead, neurotoxins, blood electrolytes, metabolites, analytes, NA+, K+, CA+, urea nitrogen, creatinine, biochemical blood markers and components, ChE, AChE, BuChe, tumor markers, PSA, PAP, CA 125, CEA, AFP, HCG, CA 19-9, CA 15-3, CA 27-29, NSE, hydroxybutyrate, acetoacetate, anti-malarial drugs such as amodiaquine, artemether, artemisinin, artesunate, atovaquone, cinchonine, cinchonidine, chloroquine, doxycycline, halofantrine, mefloquine, primaquine, pyrimethamine, quinine, quinidine, and sulfadoxine; anti-biotic drugs such as ampicillin, azithromycin, doxycycline, erythromycin, penicillin, and tetracycline; anti-retroviral drugs such as abacavir, didanosine, indinavir, lamivudine, nevirapine, ritonavir, saquinavir mesylate, zalcitabine, and zidovudine; nicotine; gonadotropin releasing hormone (GnRH), estradiol, progesterone, growth hormone, morphine, methadone, lithium, and insulin, and any other similar agents known to those of skill in the art.

The term “monitoring” is defined as testing, sampling, detecting, sensing, and/or analyzing an agent. Monitoring can either determine the presence of the agent or identify the agent itself. Moreover, monitoring includes both quantification and qualification of the agent.

The term “antigen” or “immunogen” is defined as any substance that is capable of inducing the formation of antibodies and reacting specifically in some detectable manner with the antibodies so induced. Not all antigens however, are immunogens. Examples of an “antigen” include, but are not limited to, immunogens such as viruses, bacteria, microbes, pathogens, HIV, hepatitis, anthrax, cholera, Q-fever, smallpox, tuberculosis, and any other similar biological agents or pathogens known to those of skill in the art.

The term “subject” or “patient” as used herein is defined as, but is not limited to, humans and animals.

The term “fluid” or “fluids” as used herein is meant to include, but is not limited to, blood, plasma, saliva, urine, sputum, feces, interstitial fluids, tears, sweat, water, and any other similar bodily fluids or other fluids known to those of skill in the art.

The term “label” as used herein is defined as a device that enables the quantitation and quantification of an agent. Examples of labels that can be used in connection with the present invention include, but are not limited to, chemiluminescent labels, luminescent labels, fluorescent labels, colorimetric labels, including, but not limited to, absorption, bioluminescence, and fluorescence, radiolabels, and enzyme labels.

The term “working electrode 16” as used herein is defined as, but is not limited to, an electrode that supplies the potential source for affecting oxidation and/or reduction.

The term “counter electrode 18” is defined as an electrode paired with a working electrode 16, through which an electrochemical current passes equal in magnitude and opposite in sign to the current passed through the working electrode. In the context of the invention, the term “counter electrode 18” is meant to include counter electrodes 18 that can have the dual function as a potentiometric reference electrode (i.e. a counter/potentiometric electrode). The counter electrode 18 is an electrode at which an analyte is electrooxidized or electroreduced with or without the agency of a redox mediator.

The term “amperometric electrochemical sensor” is defined as a device configured to detect the presence and/or measure the concentration of an analyte via electrochemical oxidation and reduction reactions on the sensor. These reactions are transduced to an electrical signal that can be correlated to an amount or concentration of analyte.

The term “electrolysis” is defined as the electrooxidation or electroreduction of an agent either directly at an electrode or via one or more electron transfer agents. An example of this includes, but is not limited to, using glucose oxidase to catalyze glucose oxidation creating oxidized glucose and peroxide, where the peroxide is being measured.

The term “facing electrodes” is defined as a configuration of the working and counter electrodes 16 and 18 in which the working surface of the working electrode 16 is disposed in approximate apposition to a surface of the counter electrode 18.

The term “measurement zone 28” is defined as a region of the sample chamber sized to contain only that portion of the sample that is to be interrogated during an analyte assay.

The term “non-leachable compound” or “non-releasable compound” is a compound, which does not substantially diffuse away from the working surface of the working and/or counter electrodes for the duration of an analyte assay.

The term “redox mediator” is defined as an electron transfer agent for carrying electrons between the analyte and the working electrode, either directly or via a second electron transfer agent.

The term “reference electrode 24” is defined as an electrode used to monitor and account for voltage drop due to medium resistance in amperometric sensors, and supplies a reference potential for comparison in potentiometric electrodes.

The term “second electron transfer agent” is defined as a molecule that carries electrons between the redox mediator and the analyte (See example above).

The term “sorbent material” is defined as a material that wicks, retains, or is wetted by a fluid sample in its void volume and does not substantially prevent diffusion of the analyte to the electrode.

The term “working surface 26” is defined as that portion of the working electrode, which is coated with redox mediator and configured for exposure to sample.

The term “actuator 30” as used herein is defined as, but is not limited to, a device that causes something to occur. The actuator 30 activates the operation of a valve, pump, villi, fan, blade, or other microscopic device. Typically, the actuator of the present invention affects fluid flow rates within a chamber.

The term “closed cavity 52” as used herein is defined as, but is not limited to, a sealed cavity or reservoir that contains a liquid or solid expanding mechanism 32 that is expanded or vaporized to generate expansion or actuation of a flexible mechanism 34. The closed cavity must be completely sealed in order to contain the expansion therein, and must be flexible on at least one side.

The term “expanding mechanism 32” as used herein is defined as, but is not limited to, a fluid capable of being vaporized and condensed within the closed cavity enclosed by the flexible mechanism 34. The expanding mechanism 32 operates upon being actuated or heated. The expanding mechanism 32 includes, but is not limited to: water, wax, hydrogel (solid or non-solid), hydrocarbons, and any other similar substance known to those of skill in the art. Condensation of the expanding mechanism 32 occurs when the heat, which is generated to induce expansion of the expanding mechanism, is removed by a surrounding medium such as a gas, liquid or solid. Then, once condensation occurs, contraction of the flexible mechanism 34 takes place.

The term “flexible mechanism 34” as used herein is defined as, but is not limited to, anything that is capable of expanding and contracting with the vaporization and condensation of the expanding mechanism. The flexible mechanism 34 must be able to stretch without breaking when the expanding mechanism 32 is vaporized. The flexible mechanism 34 is made of any material including, but not limited to, silicone rubber, rubber, polyurethane, PVC, polymers, combinations thereof, and any other similar flexible mechanism 34 known to those skilled in the art.

The term “heating mechanism 36” as used herein is defined as, but is not limited to, a heating device that is incorporated with the actuator 30 of the present invention. The heating mechanism 36 generates heat to induce expansion of the expanding mechanism. The heating mechanism 36 is disposed adjacent to the flexible mechanism 34 in order to turn on and off and maintaining on and off selective expansion of the expanding mechanism 32. The heating mechanism 36 can be powered using any power source known to those of skill in the art. In the preferred embodiment, a battery powers the heating mechanism 36. However, both AC and DC mechanisms are used to minimize power requirements. Generally, the heating mechanism 36 is formed of materials including, but not limited to, polysilicon, elemental metal, silicide, or any other similar heating elements known to those of skill of the art. Moreover, the heating mechanism 36 is disposed within a medium such as Si0₂ or other solid medium known to those of skill in the art.

The term “temperature sensor 38” as used herein is defined as, but is not limited to, a device designed to determine temperature. A resistive temperature sensor 38 is made from material including, but is not limited to, polysilicon, elemental metal, silicide, and any other similar material known to those of skill in the art. Thermocouple temperature sensor 38 can also be used. Typically, the temperature sensor 38 is situated within or near the heating element of the heating mechanism 36.

The terms “micro-conduit,” “microfluidic conduit,” and “conduit 40” as used herein are defined as, but not limited to, any type of tube, pipe, planar channel, conduit, or any other similar conduit known to those of skill in the art. The conduit has a wall mechanism made from material including, but not limited to, silicon, glass, rubber, silicone, plastics, polymers, metal, and any other similar material known to those of skill in the art. In one embodiment of the microfluidic valve, the conduit encompassing the micro-actuator is etched out of glass in a nearly hemispherical shape. A variety of conformations of spherically cut patterns (i.e. ⅓ of a sphere, ½ of a sphere, etc.) with differing radii and footprints are employed to provide different valving characteristics.

### PREFERRED EMBODIMENTS

The device of the present invention can be used in a variety of settings including, but not limited to, health clinics, emergency rooms, hospitals, clinical settings, home health care market, offices, work places, points of chemical exposure including possible terrorist attack sites such as in planes, trains, buildings, and any other similar settings requiring the monitoring or screening of individuals to determine and confirm exposure to various toxins and/or agents. Thus, the present invention is not meant to exclude any application outside of the medical field.

Furthermore, the present invention is well suited to test any subject including, but not limited to, employees, workers, athletes, EMS personnel, emergency first responders, and any other subject who is in need of administration of an agent for treatment of a disease or condition.

The present invention can be used to detect or treat any disease or condition. For example, the device of the present invention can be used to detect agents in order to diagnose diseases or detect the presence of toxins or pollutants. Further, the system of the present invention can be used to treat the detected disease. The following list is meant to include, but is not limited to conditions that can be treated, biological contaminants, chemical contaminants, environmental pollutants and toxins, effects of chemotherapy, levels of bilirubin, drug effectiveness, disease states, and the amount of an allergic reaction.

For example, the present invention can be use to treat diseases or conditions. Examples of such diseases include malaria, diabetes, infertility, substance addiction, dermal treatments, and other conditions as listed below.

The agent delivery device 10′ can operate in an active or in a passive manner. During active operation, a user can operate a control 70′ on the agent delivery device 10′ to acquire a sample of interstitial fluid from the membrane 60′ and perform a reaction on the captured interstitial fluid in the testing chamber 54′, and the user can monitor the results. During passive operation, the agent delivery device 10′ can automatically acquire a sample of interstitial fluid at a predetermined programmable time interval and perform a reaction in the testing chamber 54′ for a continuous monitoring of a user's interstitial fluid.

The agent delivery device 10′ can further include at least one reservoir 72′ for storing reservoir fluid being operatively connected to the membrane interface chamber 12′ and/or testing chamber 54′ by micro-conduits 40′, as shown in FIG. 38. The reservoir fluid can be any desired fluid in cleaning/calibrating the membrane interface chamber 12′ and the testing chamber 54′ such as buffer solution, calibration solution, and wash solution.

The body portion 13′ can be integrated with a patch 74′ including an adhesive backing for removable attachment to the membrane 60′, shown in FIGS. 35 and 36. The patch 74′ can optionally cover the entire body portion 13′. Adhesive can also be applied to the bottom edges 76′ of the body portion 13′ without a patch 74′ for application to the membrane 60′. Skin permeation enhancers can be applied to the adhesive such as liposomes, menthol derivatives, or glycerol derivatives to enhance the permeation of molecules through the membrane 60′.

For example, CPEs are compounds that enhance the permeation of drugs across the skin. These CPEs increase skin permeability by reversibly altering the physicochemical nature of the stratum corneum, the outer most layer of skin, to reduce its diffusional resistance. These compounds increase skin permeability also by increasing the partition coefficient of the drug through skin and by increasing the thermodynamic activity of the drug in the vehicle. Chemicals such as liposomes, menthol derivatives or glycerol derivatives can enhance the permeation of drugs through the skin.

Based on the chemical structure of penetration enhancers (such as chain length, polarity, level of unsaturation and presence of some special functional groups such as ketones), the interaction between the stratum corneum and penetration enhancers may vary which results in significant differences in penetration enhancement. Two very potent enhancers that can be considered are dimethyl sulfoxide (DMSO) and oleic acid that act by altering the level of hydration or degrading proteins and membrane lipids. Also, oleic acid incorporates into the skin lipids and disrupts molecular packing of the membrane, alters the level of hydration, and allows faster drug penetration.

Other CPEs that can be used for the enhancement of Transdermal delivery (TDD) extraction of the glucose are as follows. It has been found that polyunsaturated fatty acids PUFA-Linoleic (LA), alpha-linolenic (ALA), and arachidonic acids enhance skin permeation to a greater extent than monounsaturated fatty acids. The enhancement effects of fatty acids on penetration through the stratum corneum are structure-dependent, associated with the existence of a balance between the permeability of pure fatty acids across stratum corneum and the interaction of the acids to skin lipids. Cod-liver-oil can also be used. The enhancing effect of the marine products could generally be associated with their content of free unsaturated fatty acids. As potential skin penetration enhancers, studies have demonstrated that the permeation enhancing effect of I-menthol is significant with short lag time. The promoting activity of the ethyl ether derivative of Menthol is the greatest of all menthol derivatives. Studies have shown that this derivative is the most promising compound with the greatest action and relatively low skin irritancy. Studies have elucidated the mechanism of skin permeation enhancement and it was concluded that the increase in skin flux, up to eight times the base line, could be attributed to the effect of menthol on the skin barrier properties. Squalene was found to be a very effective skin permeation enhancer. 12% of the human sebum is composed of Squalene to which is attributed the natural moisturizing effect of the sebum. Studies also showed the skin soothing effect of Squalene. Studies concluded that glycerol monoethers derived from linear saturated fatty alcohols are very effective permeation enhancers. While specific embodiments are disclosed herein, they are not exhaustive and can include other suitable designs and systems that vary in designs, methodologies, and transduction systems (i.e., assays) known to those of skill in the art. In other words, the examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

The agent delivery device 10′ of the present invention can be used to monitor many different molecules in interstitial fluid. For example the interstitial fluid can be monitored for low molecular weight proteins to detect cancer, metabolic disease, heart function, or liver function. The low-molecular weight proteomic analysis of serum, which is believed to contain multitudes of biological markers that could provide the means for assessing an individual's health, is difficult to analyze due to the need to perform extensive fractionation to remove large proteins prior to mass spectrometric analyses. In addition, obtaining serum is necessarily an invasive procedure. Interstitial Fluid (ISF), the extracellular fluid surrounding cells, is a microcosm of human serum containing proteins and peptides at approximately thirty percent of the concentration found in serum. This was determined by applying a standardized suction technique to sample plasma proteins in dermal interstitial fluid serially for 5 to 6 days from a suction-induced skin mini-erosion. Since ISF can be obtained non-invasively, through the skin using various established techniques, and since the composition of ISF is closely related to that of serum plasma, it is an ideal body fluid to sample and monitor for biological markers.

The one “limitation” of non-invasive interstitial fluid sampling, the difficulty with which large molecules pass through the stratum corneum (SC) layer of the skin, serves as an advantage when attempting to sample and characterize the LMW components of the ISF proteome. In this respect, the stratum corneum is a natural filter allowing only the smaller LMW components to pass through while retaining the larger molecular weight components, thus eliminating the need to perform extensive fractionation of the sample. Whereas fractionation of serum to remove the high molecular weight proteins requires hours or days to perform, the agent delivery device 10′ has the potential to obtain ISF samples, containing only low molecular weight proteins, within minutes. Such an agent delivery device 10′, with the incorporation of specific marker sensors and readout circuitry, allows an individual's health status to be assessed immediately.

In a further embodiment, the micro-device 10 is a agent delivery device 10″ including a body portion 13″ housing a membrane interface chamber 12″ and a molecular delivery apparatus 82″ for delivering molecules through the membrane 60″. The agent delivery device 10″ is small, on the order of a few square centimeters or less. The agent delivery device 10″ is manufactured and made of materials essentially as described above for the micro-device 10, and integrates the circuitry, microfluidic devices, and other elements of the micro-device 10 as described above.

The molecular delivery apparatus 82″ can be at least one reservoir 72″ operatively attached to the membrane interface chamber 12″ by micro-conduits 40″. The reservoir(s) 72″ can be controlled by microfluidic valves 50″ and microfluidic pumps 47″, as described above. Agents are stored in the reservoir 72″ until the need for administration when they are released into the membrane interface chamber 12″ to be administered through the membrane 60″. Other fluids can also be stored in the reservoir 72″, such as wash fluid described above or any other suitable fluid. Additionally, the device 10 of the present invention can include numerous reservoirs 72″. The reservoirs 72″ do not have to all contain the same agent. Instead, adjacent reservoirs 72″ can contain agents that work in concert with one another. For example, one reservoir 72″ can contain the needed agent and the next reservoir 72″ can contain a skin healing agent or chemical enhancer that aids in the delivery of the needed agent. The benefit of such a configuration is a limit in potential skin irritation at the site of agent administration. Alternatively, the reservoir 72″ can be layered with different agents being encapsulated in the layers.

An electrode(s) 22″ can also be operatively attached to the membrane interface chamber 12″ for electrophoresic/iontophoretic delivery. Alternatively, other devices can be affixed to the membrane interface chamber 12″ to cause the agents to be released from the reservoir 72″. Preferably, the device is something that can administer electrons to the reservoir 72″ in order to release the agent from the reservoir 72″.

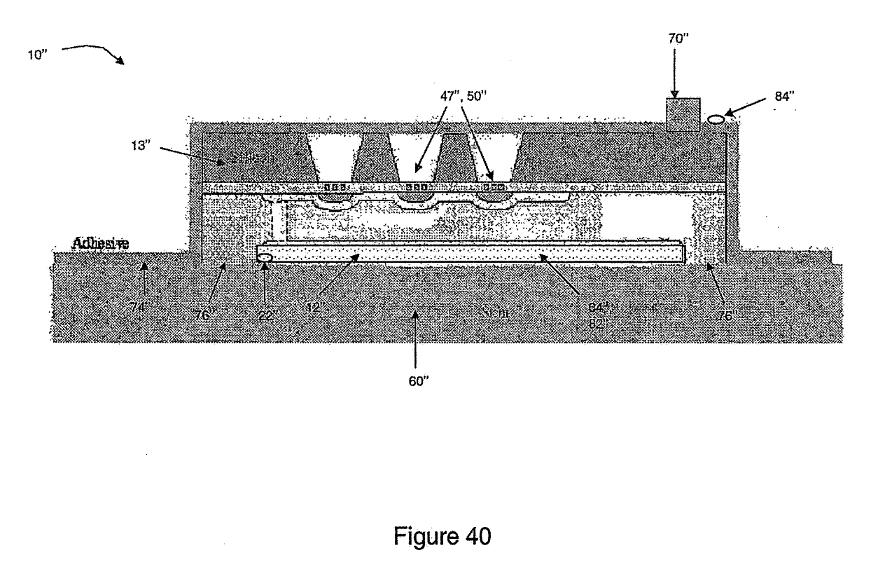

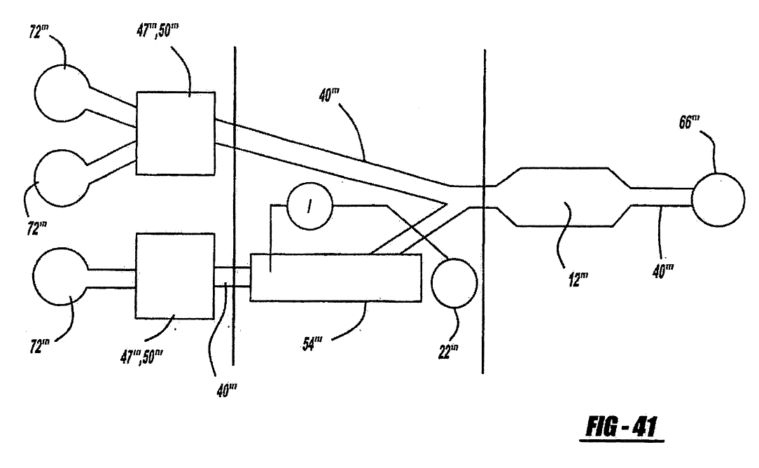

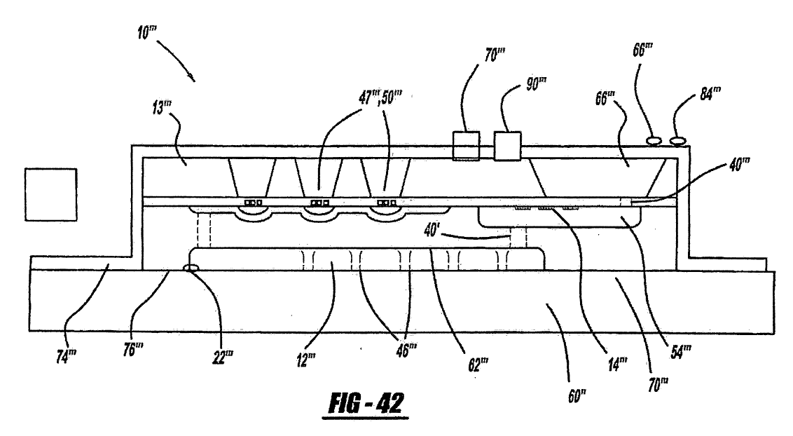

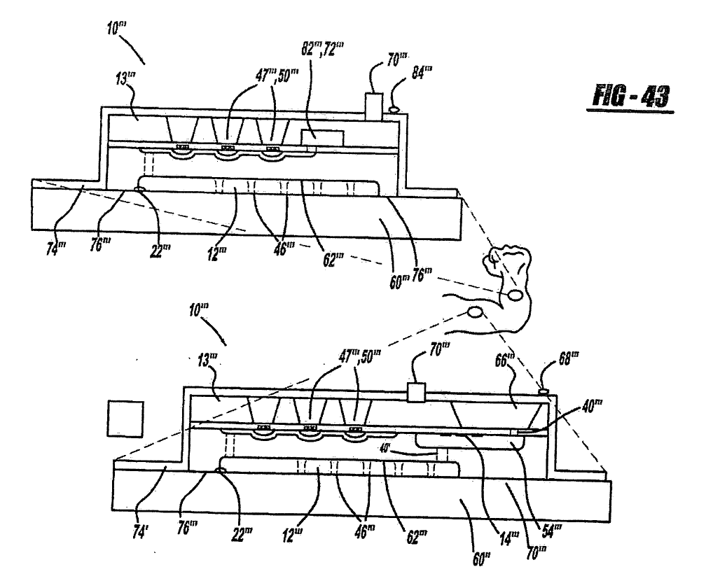

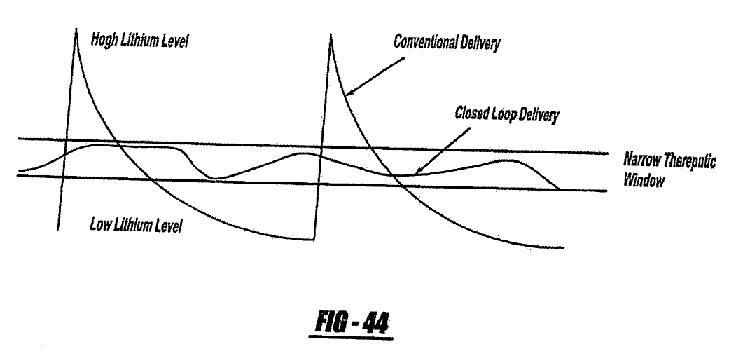

As shown in FIG. 40, the molecular delivery apparatus 82″ can also be an electrolyte polymer membrane 64″ with electrodes 22″ operatively attached, fitting inside the membrane interface chamber 12″, as described above. Embedded in the electrolyte polymer membrane 64″ are molecules which can be released by an electric current produced by the electrodes 22″, causing the molecules to be administered through the membrane 60″.

During active operation, a user can operate a control 70″ on the device 10″ to deliver molecules from the reservoir 72″. The control 70″, when activated, causes the microfluidic pumps 47″ and microfluidic valves 50″ to release molecules from the reservoir 72″, or the control 70″ causes the activation of electrodes to release molecules from the electrolyte polymer membrane 64″.

The molecular delivery apparatus 82″ can also include signal receiver 84″ to receive a telemetric signal. The signal receiver 84″ can be any suitable signal receiver 84″ and can also be operatively integrated in the device 10″ in any suitable location. The telemetric signal can activate the microfluidic pumps 47″ and the microfluidic valves 50″ to release molecules in the reservoir 72″ into the membrane interface chamber 12″ to be delivered to the membrane 60″. The telemetric signal can also activate the electrodes 22″ to stimulate the release of the molecules in the electrolyte polymer membrane 64″ to be delivered to the membrane 60″. The telemetric signal can be any signal as described above. The telemetric signal can come from a remote device such as from a handheld control, or from a main station such as a nurse's station or any other base for monitoring people.

The body portion 13″ can be integrated with a patch 74″ including an adhesive backing for removable attachment to the membrane 60″. The patch 74″ can optionally cover the entire body portion 13″. Adhesive can also be applied to the bottom edges 76″ of the body portion 13″ without a patch 74″ for application to the membrane 60″. Skin permeation enhancers, as disclosed above, can be applied to the adhesive such as liposomes, menthol derivatives, glycerol derivatives, linoleic acid, or menthone to enhance the permeation of molecules through the membrane 60″.

The agent delivery device 10″, with any of the structure described above and in active or passive delivery operation as described above, can be used to deliver molecules such as, but not limited to, nicotine for cessation of smoking, an anti-malarial agent, an antibiotic, and a gonadotropin releasing hormone for positive or negative control of fertility as further described in the examples below.

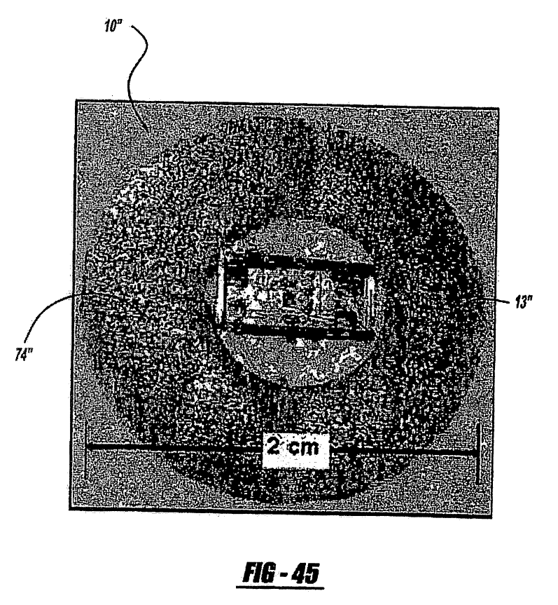

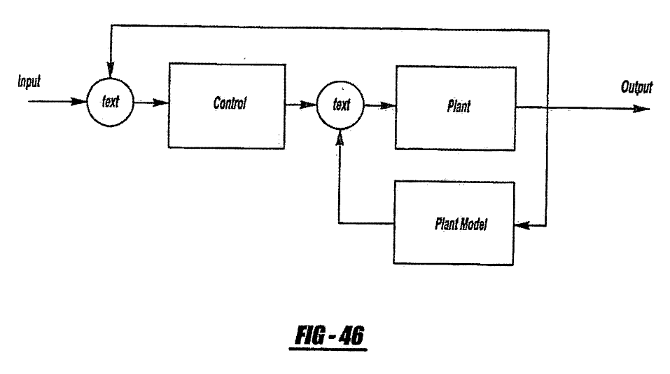

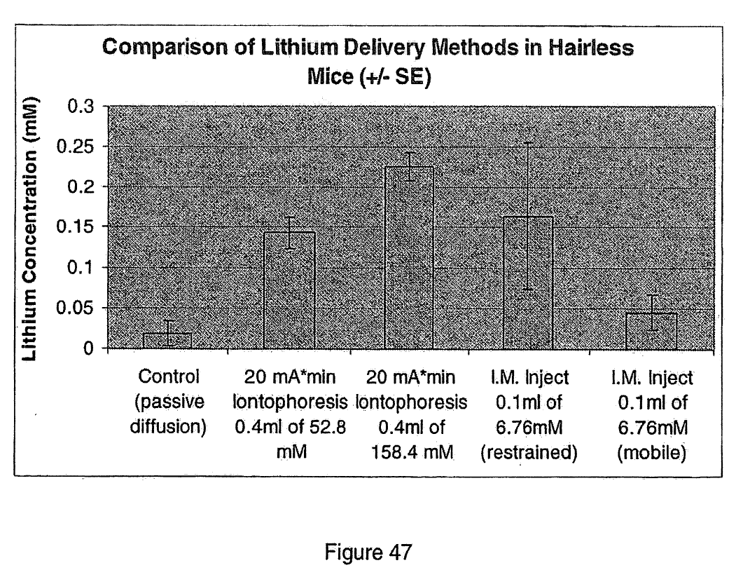

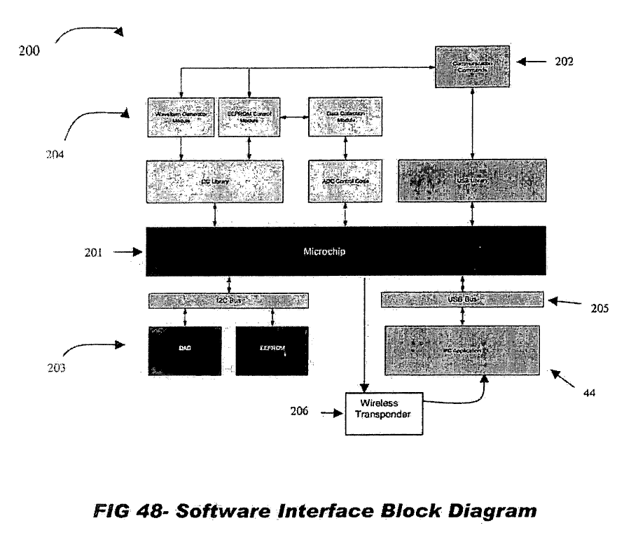

The agent delivery system 10′″ includes a transmembrane fluid capturing chamber 12′″, also called a membrane interface chamber 12′″, with electrodes 22′″ operatively integrated for capturing interstitial fluid through a membrane 60′″, a testing chamber 54′″ for detecting molecules in captured interstitial fluid, and a molecular delivery apparatus 82′″ for delivering molecules through the membrane 60′″, all essentially as described above. The agent delivery system 10′″ is small, on the order of a few square centimeters or less. The agent delivery system 10′″ is made from essentially the same materials and manufactured in the same method as described for the micro-device 10 above. The agent delivery system 10′″ is shown in FIGS. 41, 42, and 43.

The agent delivery system 10′″ can include one body portion 13′″ having the membrane interface chamber 12′″, the testing chamber 54′″, and the molecular delivery apparatus 82′″ as shown in FIGS. 41 and 42[.1]. In this configuration, the membrane interface chamber 12′″ serves as both the site for the acquisition of interstitial fluid from the membrane 60′″ and the site for delivery of molecules into the membrane 60′″. The membrane interface chamber 12′″ can include supports 46′″ or an electrolyte polymer membrane 64′″ as described above.

Alternatively, the agent delivery system 10′″ can include a body portion 13′″ having the membrane interface chamber 12′″ and the testing chamber 54′″ (essentially the agent delivery device 10′), and a second body portion having the molecular delivery apparatus 82′″ and a second membrane interface chamber (essentially the agent delivery device 10″), as shown in FIG. 43. The second membrane interface chamber has the same characteristics as the membrane interface chamber 12″ in the agent delivery device 10″ described above. In this configuration, the interstitial fluid acquisition and the delivery of molecules can occur at different places on a user's body. The membrane interface chamber 12′″ and the second membrane interface chamber can both include either supports 46′″ or an electrolyte polymer membrane 64′″, or a combination (one body portion 13′″ or 86″ has supports 46′″ and the other has an electrolyte polymer membrane 64′″). The body portion 13′″ can be placed on a membrane 60′″ at one location on the body, and the second body portion can be placed on another membrane 60′″ at another location on the body. The body portions 13′″ and 86″ can also be positioned so that one is in vivo while the other is ex vivo.

The body portion 13′″ and second body portion can be integrated with a patch 74′″ including an adhesive backing for removable attachment to the membrane 60′″. The patch 74′″ can optionally cover the entire body portions 13′″ and 86″. Adhesive can also be applied to the bottom edges 76′″ of the body portions 13′″ and 86′″ without a patch 74′″ for application to the membrane 60′″. Skin permeation enhancers can be applied to the adhesive such as liposomes, menthol derivatives, or glycerol derivatives to enhance the permeation of molecules through the membrane 60′″.

The agent delivery system 10′″ can further include at least one reservoir 72′″ for storing reservoir fluid being operatively connected to the membrane interface chamber 12′″ and/or testing chamber 54′″, and the second membrane interface chamber 88′″ by micro-conduits 40′″, as described above. The reservoir fluid can be any desired fluid in cleaning/calibrating the membrane interface chamber 12′″ and the testing chamber 54′ such as buffer solution, calibration solution, and wash solution. The reservoir 72′″ can also store molecules to be delivered. On the second body portion 86′″, at least one reservoir 72′″ stores molecules when the second membrane interface chamber includes supports 46′″.

Acquisition of interstitial fluid and delivery of molecules through the membrane 60′″ can be accomplished in an active or a passive manner. During active operation, a user can operate a control 70′″ on the body portion 13′″ to acquire a sample of interstitial fluid from the membrane 60′″ and perform a reaction on the captured interstitial fluid in the testing chamber 54′″, and the user can monitor the results. Based on the results, the user can then operate a second control 90′″ on the body portion 13′″ or on the second body portion to deliver molecules from either a reservoir 72′″ or from an electrolyte polymer membrane 64′″, as described above.

During passive operation, the agent delivery device 10′″ can automatically acquire a sample of interstitial fluid at a predetermined programmable time interval and perform a reaction in the testing chamber 54′″ for a continuous monitoring of a user's interstitial fluid. The results of the reaction can be sent from the testing chamber 54′″ to the molecular delivery apparatus 82′″ to actuate the release of molecules from either the reservoir 72′″ or from the electrolyte polymer membrane 64′″. In this manner, the agent delivery device 10′″ operates in a continuous monitoring and delivering method. The passive mode of operation is useful in the monitoring and delivery of therapeutics with narrow therapeutic windows.

Telemetry can be used in both the active and passive methods of operation. The testing chamber 54′″ can include a signal transmitter 68′″ as described above. The molecular delivery device 82′″ also includes a signal receiver 84′″ as described above. The signal transmitter 68′″ and the signal receiver 84′″ operate essentially as described above, acquiring a sample and transmitting a signal with data to a receiver, and receiving a signal with data to activate delivery of molecules, and optionally transmitting/receiving signals to/from a main station.

The telemetry in the agent delivery device 10′″ can also operate in an additional method of a feedback system for real-time monitoring. The feedback system causes interstitial fluid to be obtained periodically from the membrane interface chamber 12′″. Then, the captured interstitial fluid is tested in the testing chamber 54′″. A signal is generated based on the data from the testing chamber 54′″. This signal of feedback from the testing chamber 54′″ is sent from the signal transmitter 68′″ to the signal receiver 84′″, where it is interpreted and thereby actuating the release of molecules by the molecular delivery apparatus 82′″ for administration through the membrane 60′″. The feedback system can operate with one body portion 13′″ and also with the second body portion. When the second body portion 86′″ is included, the signal from the signal transmitter 68′″ on the body portion 13′″ travels to the signal receiver 84′″ on the second body portion. Using a feedback system provides higher control in dosing and response as shown in FIG. 44, especially with drugs having a narrow therapeutic window (such as lithium), and is advantageous over other methods of drug delivery.

The agent delivery device 10′″ can automatically dispense molecules at a predetermined programmable time interval in a pulsatile release manner. In other words, molecules can be automatically released in pulses from the reservoir 72′″ or the electrolyte polymer membrane 64′″ can be automatically stimulated by the electrodes to release molecules in pulses. Pulsatile delivery can be used with telemetry and a feedback system. For example, the membrane interface chamber 12′″ can acquire interstitial fluid, test it in the testing chamber 54′″, the signal transmitter 68′″ can send a signal to the signal receiver 84′″, which actuates the release of molecules by the molecular delivery apparatus in a pulsatile manner.

For some types of drugs, it is preferred to release the drug in “pulses,” wherein a single dosage form provides for an initial dose of drug followed by a release-free interval, after which a second dose of drug is released, followed by one or more additional release-free intervals and drug release “pulses.” Pulsatile drug delivery is useful, for example, with active agents that have short half-lives and must be administered two or three times daily, with active agents that are extensively metabolized presystemically, and with active agents which lose the desired therapeutic effect when constant blood levels are maintained. These types of agents have pharmacokinetic-pharmacodynamic relationships that are best described by a clockwise “hysteresis loop.” A drug dosage form that provides a pulsatile drug release profile is also useful for minimizing the abuse potential of certain types of drugs, i.e., drugs for which tolerance, addiction and deliberate overdose can be problematic and creates a more natural drug delivery. Further, pulsatile delivery is advantageous for drugs that have a narrow therapeutic window, usually requiring close monitoring and a smaller dose at a more frequent interval. The amount of drug in the body can be controlled easier with pulsatile delivery, maintaining effectiveness while reducing side effects. Several drugs having a narrow therapeutic window include, but are not limited to, levothyroxine, phenytoin, warfarin, theophylline, lithium, digoxin, and 5-fluorouracil.

Pharmaceutical companies employ a variety of approaches for overcoming the problem of pre-systemic elimination in oral drug administration. Included among these approaches is the use of physical and chemical agents to delay drug metabolism, alternate delivery routes to bypass hepatic metabolism and pulsatile delivery systems, mainly in the form of layered pills or capsules for oral intake, to control the rate of drug release. Despite the efforts necessary to develop these techniques, they have failed to address the problems associated with the continuous and/or oral administration of drugs. The agent delivery device 10″ can overcome previous techniques by providing more accurate pulses of molecules. With a feedback system, the agent delivery device 10′″ can also closely monitor molecule levels in the body and give pulses of required molecules more accurately when needed.

The agent delivery device 10′″ can be used for many different applications such as, but not limited to, analyzing captured interstitial fluid for melatonin and delivering molecules including melatonin for treating a sleeping disorder, analyzing captured interstitial fluid for glucose and delivering molecules including insulin for treating diabetes or stress, analyzing captured interstitial fluid for lithium and delivering molecules including lithium for treating a psychological disorder, delivering molecules including butylcholinesterase or atropine for acute treatment of chemical warfare agents, or delivering hormones, buserelin, methylphenidate, or mecamylamine. Several of these applications are further described in the examples below.

For example, glucose concentration in blood can be used to determine metabolic status as well as to assess the degree of psychological and physical stress experienced by the individual, by providing indications of their homeostatic condition and providing evidence of stress.

In addition to lithium and other psychotic drugs, such as valproate and haloperidol, the device can non-invasively monitor, in real-time, hundreds of other biological markers such as blood electrolytes, blood ions, glucose, biologically active substances, pharmacological drugs, drugs of abuse, pesticides, hormones, etc. Further, it is possible to customize the system to automatically deliver different types of medication in precise amounts. For example, one application allows insulin-dependent diabetics to closely regulate their blood sugar and maintain a healthy state of euglycemia. With a focus on controlled lithium delivery and the potential for many other applications, the LDMS revolutionizes how diseases are treated today and make proper regulation an attainable goal for everyone.

The device 10 of the present invention can also be used for the treatment of diabetes, manic depression, anxiety disorders, smoking cessation, antibiotic application, or hormonal therapy for fertility, infertility, growth disorders, sleep disorders, etc. or application in the cosmetic industry to remove facial skin wrinkles, acne scars, and other cosmetic treatment to facial features and to return plasticity to aging or full thickness burn damaged skin. The system of the present invention can be utilized to target and induce the formation of collagen, in the appropriate orientation and at a high rate of deposition, in a non-invasive manner. As a result, the skin's elasticity and plasticity can be improved and/or restored.

In treating the skin, the device 10 of the present invention is capable of laying a scaffold of precursor substrates in an individual. The scaffold can be established in the epidermis, dermis, subcutaneous fat, or in any other layer within the body of an individual. The scaffold is defined as a supporting framework of precursor substrates wherein the precursor substrates are aligned and/or oriented in a manner that aids in the formation of collagen. Alignment and/or orientation of precursor substrates occur via electromagnetic stimulation. The electromagnetic stimulation increases the growth rate and control of orientation of the newly formed collagen molecules.

While specific embodiments are disclosed herein, they are not exhaustive and can include other suitable designs and systems that vary in designs, methodologies, and transduction systems (i.e., assays) known to those of skill in the art. In other words, the examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Malaria

A wearable anti-malarial pulsatile administration device (AMPAD) that delivers anti-malarial drugs in a transdermal, pulsatile manner was developed. The AMPAD includes a micro-iontophoresis system, constructed using MEMS and CMOS technologies, and a polymer matrix electrolyte reservoir that contains the drug. The system delivers precise square wave pulses of antibiotic through the skin to increase the efficacy of treatment, as well as compliance to anti-malarial prophylaxis, by eliminating the side effects that result from oral administration.

Polymer matrix electrolytes have been shown to be ideal for storage and delivery of molecules, such as lithium and lidocaine, since the polymers trap the molecules and release them only when a current is applied to the matrix. The microcircuitry, manufactured using CMOS technology, is integrated into a single silicon chip. The device is powered by a thin film battery, built into the protective casing that surrounds the unit, providing a self-contained device the size of a band-aid. The protective casing as well as the entrapment of the molecule in a solid matrix, which is released only when current is applied, provides a fail-safe mechanism such that in the event of damage to the device, the patient can be protected from inadvertent exposure to the drug. Such a device is needed to increase compliance, reduce the costs, and increase the efficacy of antibiotic therapy.

None of the prior art methods of transdermal delivery are very efficient (requiring large patches for an effective dose) or are capable of delivering an anti-malarial agent/antibiotic in a pulsatile manner, as the agent delivery device described above. To aid in the delivery of hydrophobic antibiotic molecules, the agent delivery device uses an electrolyte polymer membrane, to trap the molecule and release it when current is applied. The agent delivery device is a wearable transdermal patch that incorporates a micro-iontophoresis system, constructed using MEMS and CMOS technologies, and an electrolyte polymer membrane containing sufficient drug to deliver precise square wave pulses of antibiotic to increase the efficacy of treatment, as well as compliance to anti-malarial prophylaxis, by eliminating the side effects that result from oral administration.

There are a number of anti-malarial drugs currently in use. For the best protection against malaria, mefloquine, doxycycline, chloroquine, atovaquone/proguanil, or primaquine are commonly prescribed. However, the number of effective drugs available to treat malaria is small and the rate at which resistance is growing is outpacing the development of new antimalarials. The main obstacle to malaria control is the emergence of drug-resistant strains of the parasite P. falciparum, the deadliest of all the malaria pathogens.

The lipophilicity of anti-malarial drugs makes them good candidates for transdermal absorption. Moreover, the use of a pulsatile transdermal anti-malarial drug delivery system provides a means to decrease or eliminate the development of resistance to these drugs. The technology combats both the problem of resistance and the problem of non-compliance to oral administration of antibiotics.

Researchers have been investigating the transdermal delivery of various anti-malarial drugs including the following. Triclosan is widely used as an anti-bacterial agent and it has recently been demonstrated that this compound has anti-malarial properties. Its high lipophilicity makes it a potential candidate for delivery across the skin. It was determined that a simple transdermal patch could deliver a therapeutic in vivo dose of primaquine across full-thickness excised human skin, with possibilities for the treatment and prophylaxis of Plasmodium vivax, P. ovale and P. falciparum forms of malaria. Researchers have accumulated data that suggests (1) significant amounts of doxycycline, a potent anti-malarial drug, can be administered into and across human skin; (2) Migliol 840 is a potentially useful enhancing vehicle; and (3) significant amounts of drug were delivered transdermally. In the first 3 hours following introduction of erythromycin lactobionate, 1.85 mg/cm² crossed human epidermis. Given that a dose of 50 mg may exert prokinetic effects in vivo in man, increasing the patch size to approximately 28 cm² should provide therapeutic levels of drug within 3 hours.

To aid in the delivery of hydrophobic antibiotic molecules, the present invention uses an electrolyte polymer matrix, to trap the molecule and release it when current is applied. Polymer electrolyte films have been shown to be useful for electrotransport of drugs, e.g., lidocaine hydrochloride and lithium chloride. The polymers are cast from solutions of poly(etheleneoxide) (PEO) and various drug salts using either water (for hydrophilic molecules) or an acetonitrile/ethanol mixture (for hydrophobic molecules) as the casting solvent. AC impedance analysis demonstrates that the conductivity of the films vary between 10⁻⁶ and 10⁻³ S cm⁻¹, depending on the salt, casting solvent, and temperature.

In addition to antibiotic delivery, the device of the present invention can also be used for the delivery of other hydrophobic and hydrophilic drugs and hormones. The device's ability to deliver drugs in a pulsatile manner has proven to have advantages over continuous delivery. As previously indicated, the pulsatile delivery of drugs increases their effectiveness while simultaneously decreasing side-effects. The device's ability to deliver drugs in a transdermal manner has proven to have advantages over oral administration, including the need to address pre-systemic elimination. Pharmaceutical companies employ a variety of approaches for overcoming the problem of pre-systemic elimination in oral drug administration. Included among these approaches is the use of physical and chemical agents to delay drug metabolism, alternate delivery routes to bypass hepatic metabolism and pulsatile delivery systems, mainly in the form of layered pills or capsules for oral intake, to control the rate of drug release. Despite the efforts necessary to develop these techniques, they have failed to address the problems associated with the continuous and/or oral administration of drugs.

To meet this objective, an anti-malarial antibiotic was incorporated into a polymer electrolyte and the polymer was cast into a mold the size of a band-aid, approximately 2 cm in diameter. Polymer electrolytes are solid-like materials formed by dispersing a drug in a high molecular weight, lipophilic polymer. In essence, the molecule is trapped within the polymer until the application of an electric current. Application of electric current causes the porosity and diameter of the pores of the polymer to increase, hence providing controlled release of the drug. The technology allows molecular concentrations as high as 4 molar to be incorporated into the matrix.

The patch was applied to human skin samples using an in vitro iontophoresis apparatus to measure the flux of antibiotic that crosses the skin after application of electric current to demonstrate that enough transdermal antibiotic is delivered transdermally to mimic serum levels achieved by oral administration.

Films of PEO (RMM: 4,000,000, Aldrich) mixture were prepared using a standard solvent casting technique for the preparation of polymer electrolyte films. The compositions were in the form PEOn:antibiotic (where n=10 or 20). This represents the molar ratio of the ethylene oxide (EO) repeating unit to antibiotic. PEO10:antibiotic represents 1 molecule of antibiotic associated with 10 EO units. For each preparation, 1 g of PEO was used and the mass of antibiotic to be used was calculated by dividing the molecular mass of the antibiotic by the molar ratio of 10 and the molecular mass of EO repeat unit (i.e. 44).

The calculated mass of antibiotic was then added to 1 g of PEO in 50 mL of distilled water (for hydrophilic molecules) or acetonitrile:ethanol (for hydrophobic molecules) and stirred until complete dissolution. The mixture, which was a viscous solution, was then cast into polystyrene 2 cm diameter culture dishes. Before the polymer had cured, a loop of platinum wire was inserted into the solution such that it was firmly held in place by the cured polymer. The solution was then covered and the solvent was allowed to evaporate at room temperature. The film was then peeled from the well and stored in a sealed plastic bag over silica gel in a desiccator.

A pressure sensitive adhesive (PSA), such as an acrylic emulsion, was applied to the bottom of the patch to provide a tight seal between the polymer and skin. New polymer adhesives have become available to advance transdermal technology. The polymers have been modified to improve solubility and drug diffusion with little change in adhesive and cohesive properties. 3M's Latitude™, and CORPLEX™, both of which are polymer adhesives, has a versatile range of properties for water sorption and adhesion to moist skin.

The delivery electrode was incorporated into the polymer-antimalarial matrix, which was placed on top of the skin in the donor compartment of the device, while the return electrode was inserted into the receptor compartment.

Construct electrolyte polymer delivery pad: To measure electrochemical degradation that was caused by iontophoresis, thin layer chromatography cab be used. An initial experiment was performed to determine the sensitivity of this method and the migration pattern of primaquine. Silica gel plates were used to spot 50, 5, 0.5, and 0.05 μg of primaquine. n-butanol:acetic acid:water (5:3:2) was used as the solvent and the chromatography was run for four hours at room temperature.

It is apparent from this experiment that the system permits any degradation products, due to electrochemical degradation, to be visualized easily since all degradation products are of lower molecular weight and would appear as spots below the primaquine spots pictured above. However, a better developing reagent is needed, such as Dragendorff's reagent since the iodine vapor also colors the TLC silica gel and reduces the resolution contrast considerably.

To quantify the amount of primaquine that can be delivered transdermally, the absorbance of UV light by primaquine was investigated. Using a BioTek Synergy HT plate reader, a full absorbance spectrum was run using two different primaquine concentrations.

The absorbance spectrum shows an absorbance peak at 340 nm wavelength. The wavelength was used to measure the dose response of primaquine. A standard curve was prepared using concentrations of 0.03125-0.5 mg/ml, in duplicate. The absorbance at 340 nm was plotted vs. primaquine concentration.

Researches have found that 10 mg of primaquine can be delivered, transdermally, within 24 hours to achieve therapeutic plasma concentrations. Approximately 5% of the 10 mg dose is delivered passively each hour. The use of iontophoresis increases the delivery rate and transdermal flux.

Since the receptor compartment of the in vitro transdermal diffusion device is 5.0 ml, and assuming the maximum amount of primaquine delivered is 10 mg, the maximum concentration in the receptor compartment is 2.0 mg/ml. Estimating that 10 percent of the total amount of primaquine is delivered per pulse of current, direct measurement of the receptor compartment absorbance at 340 nm gives reliable primaquine concentrations using the same standard curve.

Casting of electrolyte polymer-primaquine matrix: A drug patch was prepared and tested for the ability to release the drug when current is applied.

The patch was prepared by casting PEO (polyethylene oxide, the electrolyte polymer) into a polydimethylsiloxane (PDMS) polymer mold and allowing it to dry at room temperature. The mold was prepared by casting 200 ml of a two part PDMS (Sylgard 184) mixture into a Petri dish containing a Teflon wafer at the bottom and surrounded by a foil sleeve. After curing at 90° C. for 30 minutes, a 1 cm cork borer was used to bore a hole into the PDMS and create a mold. This type of mold is needed since the polymer-drug mixture sticks to most surfaces. The Teflon-PDMS mold allows the patch to be released from the mold easily.

A mixture of polyethylene oxide (PEO) and primaquine was made by first dissolving 0.1 g of PEO in 10 ml of distilled water. The mixture was heated to 100° C. until dissolved. After cooling, 0.102 g of primaquine was added and shaken on a Vortex mixer until dissolved. 2.5 ml of the PEO-primaquine mixture was added to the mold and the solution was allowed to dry at room temperature. A platinum electrode wire loop was inserted into the mold along with the PEO-drug mixture.

Periodically, over the course of a week, the solution was topped off with more of the PEO-primaquine mixture until a total of 8.0 ml was added and dried. The result was a PEO-primaquine patch containing 80 mg of drug. After drying, the patch was coated with a silicone pressure sensitive adhesive (BIO-PSA 7-4602), a hydrophobic adhesive that can be used to attach the patch to the skin, to determine the device's permeability to the drug.

To accelerate the drying time, it was thought that a better system would be one that provided a large surface area during drying. In this manner, the patches could be cut using the cork borer after the polymer-drug matrix had thoroughly dried. To do this, a second mold was created by coating a thin layer of PDMS onto the bottom of a 100 mm Petri dish and adding 100 ml of the PEO-drug mixture the plate, filled to the brim.

Prepare iontophoresis systems: To test the functionality of the electrolyte polymer to release primaquine when current is applied, the patch was suspended on the surface of a balanced salt solution while current was applied using the Phoresor II iontophoresis system. A 300 ohm resistor (to mimic the resistance of human skin) was soldered to a section of platinum wire and placed into the salt solution. The positive electrode was connected to the patch electrode and the negative electrode was connected to the resistor. Since primaquine is a positively charged molecule, migration is toward the negative electrode. A current dose of 80 mA*min was applied to the patch and 100 μl aliquots were sampled every 10 min.

After the electrodes were connected to the patch and placed in the reservoir, 100 μl samples were collected every 10 minutes. No current was applied for the first 20 minutes to determine if there was passive release of the drug. At 20 minutes, an 80 mA*min current dosage was applied to the patch and samples were collected. A “halo” of drug was apparent in the receptor compartment after 10 minutes of iontophoresis. Before sampling the receptor compartment, the contents were thoroughly mixed by aspirating the liquid several times with a pipette.

The 100 μl samples were placed in the well of a microtiter plate (2 samples per time point) and read at a wavelength of 340 nm. Since only a balanced salt solution was used in the receptor compartment, the only ultraviolet absorbing compound present is primaquine. The results indicate that only a minimal amount of Primiquine was released prior to application of current. After the onset of iontophoresis, the absorbance increased four fold.

The data from these experiments indicate the following: 1. The formulation used for the PEO-primaquine patch is suitable for fabricating the transdermal patch; 2. The pressure sensitive adhesive used is permeable to the drug and allows the flow of current; 3. There is minimal passive diffusion of primaquine from the patch with no current applied; 4. There is significant delivery of drug after the current is applied.

Casting of electrolyte polymer-primaquine matrix: To accelerate the drying time of the casting process, a mold was created by coating a thin layer of PDMS onto the bottom of a 100 mm Petri dish and adding 100 ml of the PEO-drug mixture the plate, until the plate is filled to the brim. This mixture was placed in the dark to dry for 1 week before cutting individual patches with a 1 cm cork borer.

While the polymer was still moist, platinum wire loops were placed in the polymer to dry. The loops can also be inserted after drying by placing 100 μl of dH₂0 over the area to solubilize the surface of the polymer/drug. After drying, the loop is firmly attached to the patch. A 1 cm cork borer was used to cut out individual patches for testing.

Since 1 gram of primaquine was added to the 100 mm plate (radius=5 cm, area=78.5 cm2), the amount of drug in the plate after casting and evaporation was 1 gram/78.54 cm2. Given that the patches were cut using a 1 cm cork borer (radius=0.5 cm, area=0.785 cm2), the concentration of drug in the patches was 1/100^{th} the total amount of primaquine in the mold or 10 mg of primaquine per patch.

Perform experiments to determine pulse delivery efficiency of antimalarial: Three types of skin membranes can be prepared for in-vitro transdermal delivery experiments: epidermal membranes with a thickness of approximately 0.1 mm, are prepared by heat, chemical, or enzymatic separation; split-thickness skin with a thickness of 0.2-0.5 mm are prepared using a dermatome; and full-thickness skin with a thickness of 0.5-1.0 mm. Since the main barrier to drug delivery for the skin is located in the stratum corneum, all three membrane types have been used for absorption studies. Moreover, since the capillary network begins just below the epidermis and is contained throughout the dermis, in-vitro flux determinations using full thickness skin may yield an over-estimate of the time required for the drug to reach the capillary network, since the time measured is the time needed to entirely bypass the capillary network and reach the receptor compartment of the diffusion cell.

For the initial transdermal studies and since most of the barrier function is contained in the stratum corneum, epidermal membranes (containing the stratum corneum and epidermal layers) were used for these experiments.

Human skin was obtained from the National Disease Research Interchange (NDRI), procured from an abdominoplasty procedure. The subcutaneous fat was removed using blunt dissection with a scalpel. The skin sample was placed in distilled water at 60 C for 1 minute to loosen the epidermal layer. Using forceps, the epidermal layer was removed by teasing it away from the dermis.

To visualize the integrity of the membrane and assure that there were no visible holes or tears, the membrane was viewed microscopically after placement in the permeation device using an inverted phase contrast microscope. In this manner, each epidermal membrane was examined before proceeding with the experiment to ensure its integrity.

Using a 1 cm cork borer, membrane discs were cut and inserted into a Mattek permeation device. The primaquine patch, fabricated and coated with adhesive as described in previous reports, was applied to the membrane and the donor compartment was attached and secured. The assembly was placed into a 25 mm culture dish containing 5.0 ml of phosphate buffered saline (PBS) at pH 7.4.

To determine the amount of passive diffusion, no current was applied to the device for 1 hour, at which time the first 200 μl sample (in duplicate) was taken from the receptor compartment and placed into the wells of a 96 well microtiter plate. A current dose of 80 mA*min at a current level of 4 mA was then initiated and samples were collected at 10 and 20 minutes. Immediately after the first iontophoresis dose was completed, a second 80 mA*min current dose also at a current level of 4 mA was applied and at the end of this dose, samples were collected. After 20 minutes with no current applied, the final samples were taken to again determine passive diffusion. The experiment was repeated three times with three membrane samples and three separate patches.

After completion of the experiment, a standard curve was prepared and 200 μl samples were placed into the microtiter plate. The UV absorbance at 340 nm was measured using a BioTek Synergy HT plate reader. The concentration of UV absorbing primaquine in the receptor compartment was determined by extrapolation to the standard curve, corrected for volume at the time of sampling. Minimal passive diffusion was observed before and after iontophoresis.

Unlike passive delivery patches, that increase the flux of drug delivery as the patch size increases, electrotransport is a function of the current applied and is independent of the size of the patch. For this reason, a smaller patch is better for pulsatile iontophoretic delivery since the amount of drug delivered between pulses is minimized.

Perform experiments to determine maximum deliverable dosage of antimalarial and stability: To determine the stability of the primaquine molecule after exposure to iontophoresis, the receptor compartment from one of the delivery experiments was dried down under nitrogen and reconstituted with 100 μl of dH₂O. Primaquine standards were prepared at 50 μg/10 μl, 5 μg/10 μl, 0.5 μg/10 μl, and 0.05 μg/10 μl. 10 μl samples were added to a silica gel plate with UV indicator. The TLC was developed using n-butanol:acetic acid:water (5:3:2) as the solvent and the chromatography was run for four hours at room temperature. The photograph shows a broad band for the receptor compartment contents indicating that a) intact primaquine is present, and b) there is more than one species of molecule present.

To prepare the patches, the previous casting method was modified by using smaller PDMS coated Petri dishes (35 mm) and drying in an oven at 60 C for 5 hours to reduce the drying time. This method gave patches that appeared less oxidized and retained the bright orange color of the primaquine.

A modified casting method for preparing the primaquine patches has been developed. 35 mm Petri dishes coated with PDMS were prepared and cured. To the Petri dish was added 15 ml of primaquine-PEO containing 1 g of primaquine and 2 g of PEO in 100 ml of distilled water. Platinum wire coils were inserted into the patch after 4 hours of drying time. A 1 cm cork borer was used to cut the patches from the mold.

Since 15 ml of primaquine-PEO containing 1 g/100 ml of primaquine is added to the mold, 0.15 g of total drug is distributed across the area of the plate. For the 35 mm Petri dish (radius=1.75 cm, area=9.62 cm2) the distribution of drug is 0.15 g/9.62 cm2=15.6 mg/cm2. Therefore, with a patch size of 1 cm (radius=0.5 cm, area=0.785 cm2), 12.25 mg of primaquine is contained in each patch.

After cutting the patches from the mold, the platinum wire was fed through a holder fashioned from the end of a 1 cc syringe needle plunger with a hole drilled through its length.

To hold the patch and mouse in place during the animal studies, a small rodent restrainer has been modified with Plexiglas brackets that attach to the base of the restrainer.

For the studies, mice were exposed to various currents and current dosages to determine the maximum dosage to deliver primaquine without harm to the animal. After exposure, the animals were sacrificed by decapitation and trunk blood can be collected. This was performed at 15 minutes, 30 minutes, and 60 minutes after exposure to determine the delivery profile. Sham mice, receiving no iontophoresis treatment were used.

For the extraction of primaquine and its metabolite carboxyprimaquine from whole blood, the procedure of Ward et al. was followed with some modifications. Briefly, 2 ml of 25% ammonia solution (specific gravity 0.91) was added and vortex mixed for 2 minutes. The mixture was extracted with n-hexane-ethylacetate (3.5:0.5, v/v) and centrifuged at 1000 g for 10 minutes to separate the phases. The organic phase was separated and evaporated to dryness under nitrogen. The residue was reconstituted with 25 μl of n-hexane-ethylacetate (3.5:0.5, v/v). The samples were run using silica-gel thin layer chromatography to qualitatively determine the presence or absence of primaquine in the blood for the patch treated and untreated animals, respectively.

Results and Technical Feasibility: In summary, since the therapeutic dosage of Primaquine for the treatment of malaria is 0.03 μg/ml, and assuming approximately 5 liters of blood in an adult human, it is necessary to deliver 150 μg of the drug to reach the therapeutic level. Research of the literature reveals Primaquine half-life values ranging from 3 to 9 hours. Therefore, 75 μg is required to be delivered every 3 to 9 hours to maintain the therapeutic level of the drug. Since 160 μg can be delivered in 40 minutes using electrotransport, the proposed AMPAD device is a viable alternative for maintaining therapeutic levels of the drug, avoiding the oral administration route and associated side effects and increasing compliance to the treatment regimen in soldiers and others. In addition, the ability to deliver square wave pulses of the drug reduces the development of resistance.

### Example 2

### Nicotine

Current transdermal patches deliver nicotine in a passive manner and are not capable of pulsatile delivery. Nicotine gum, inhalation devices and lozenges deliver nicotine in much the same manner. The nicotine spray delivers a pulse of nicotine that resembles the same delivery pattern as that of smoking a cigarette, but can only deliver half the amount of nicotine. Decreasing the dosage of spray during a smoking cessation regimen requires a different formulation of spray, containing smaller and smaller amounts of nicotine. This complicates the ability to deliver serially decreasing doses of nicotine as are typically utilized in addiction withdrawal programs. In addition, since the rate of delivery is completely controlled by the patient, it is possible that the spray can be over-used.

Current nicotine delivery patches rely on the passive diffusion of nicotine through the skin and into the fluid that surrounds the cells beneath the skin (interstitial fluid). From there, the nicotine diffuses into the capillary network, enters the blood stream, and is delivered to the brain. The nicotine is contained in a textile fiber material within the patch and nicotine is delivered continuously, as long as the patch is worn. This method of delivery fails to mimic plasma nicotine levels produced by cigarette smoking since it is not pulsatile and does not deliver the same level of nicotine.

The use of passive diffusion nicotine patches as part of a smoking cessation regimen has proven to be ineffective. In fact, no advantage for nicotine replacement therapy (NRT) was observed in either the short or long term for nearly 60% of California smokers classified as light smokers (<15 cigarettes/day). Since becoming available over the counter, NRT appears no longer effective in increasing long-term successful cessation in California smokers.

The agent delivery device, with incorporated microfluidic pumps and valves, provides the capability to deliver nicotine in a truly pulsatile manner by a less than 2 cm² patch. By means of the microfluidic pumps and miniature reservoirs, various levels of nicotine can be introduced into the reservoirs for iontophoretic transdermally delivery. The “on state” can be followed by an “off state” wherein the nicotine is completely emptied from the reservoir and replaced with normal saline, or left empty, and the iontophoresis electrode is turned off.

In this manner, true square-wave pulses of nicotine can be delivered. Unlike current transdermal nicotine patches, which do not have the capacity to remove the nicotine from the system other than by removing the patch, the agent delivery device is fully automated, programmable, and can deliver nicotine in a pulsatile manner. The nicotine pulses can be continuously decreased during the entire cessation regimen. Since the plasma nicotine profile more closely resembles that obtained while smoking a cigarette, the agent delivery device is more effective, thus increasing the likelihood that the full cessation regimen can be followed.

The agent delivery device can be worn for one day during waking hours (removed at night, applied in the morning). Depending on the most effective cessation regimen, a series of agent delivery devices can be manufactured with serially decreasing dosages of nicotine. The “Day 1” delivery dosage for each pulse can be automatically decreased by a minimal amount throughout the day with the ending dose being equal to the starting dose of the following day “Day 2” agent delivery device, thus providing the ability to slowly and serially decrease the nicotine dosage throughout the treatment period. The interval between delivery of nicotine can also be modulated throughout the day.

The storage volume of the nicotine solution is not limited to the 120 μl volume of the reservoir. Soft polymer PDMS reservoirs can be constructed and bonded to the silicon chip to easily provide 1.0-2.0 ml storage volumes. With an initial nicotine concentration of 50 mg/ml (maximum solubility), the 20 μl membrane interface chamber can contain 1 mg of nicotine. The membrane interface chamber is continuously replenished during the pulse period using the microfluidic pumps, thereby providing a constant concentration of nicotine in the membrane interface chamber. In this manner, current and time are the limiting variables. For example, a pumping rate of 20 μl per minute can make 5 mg available for delivery within a five minute pulse, thereby requiring only 20% delivery efficiency to equal the required 1 mg dose. A storage volume of 2.0 ml can supply sufficient nicotine for at least 25 five minute pulses, or 50 two and a half minute pulses (truly any combination or permutation) to be delivered throughout the day.

The membrane interface chamber can be emptied and filled with an isotonic buffer or saline solution between pulses. The entire patch can be covered with a backing layer of polyester film, which also houses a battery, similar to existing passive dermal patches. The nicotine solution can also be used with an electrolyte polymer membrane as described above that can prevent “leakage” both within and outside the patch. The electrolyte polymer membrane can be stimulated by electrodes to release the nicotine solution in pulses.

Cyclic voltammagrams indicted that nicotine is oxidized at voltages approaching 1 volt. The half-cell containing nicotine can be kept at a potential below 0.7V.

Polymer matrix electrolytes have been shown to be ideal for storage and delivery of molecules, such as lithium and lidocaine using iontophoresis. Polymer electrolytes are solid-like materials formed by dispersing nicotine in a high molecular weight polymer. In essence, the molecule is trapped within the polymer until the application of an electric current. Application of electric current causes the porosity of the polymer to increase, hence providing controlled release of nicotine. This technology allows molecular concentrations as high as 4M to be incorporated into the matrix. The use of polymer electrolytes to deliver nicotine can simplify the agent delivery device considerably since it can eliminate the need for reservoir and pumps. CMOS circuitry can control the amplitude and duration of the nicotine transfer in order to deliver precise amounts of nicotine. This can also provide a secondary fail-safe mechanism in case of trauma to the patch, or failure mode operation since transdermal delivery of nicotine only occurs when current is applied.

Polymer electrolytes are ionically conducting polymers that are composed essentially of solutions of ionic salts in heteropolymers, such as poly(ethylene oxide) (PEO). PEO is a semicrystalline solid with a high proportion of crystalline regions distributed in a continuous amorphous phase, which means the PEO is a solid at room temperature (tm=65 C and Tg=−60° C., thus it has structural integrity) and the PEO chains in the amorphous regions have a sufficient degree of segmental mobility, permitting ion transport. The amount and state of amorphous regions of polymer is therefore crucial to its functioning as a polymer electrolyte, which can be altered by many factors, including the type and amount of added ions (including medicinal drugs) and the method by which the polymer electrolyte is formed.

As its low molecular weight analogs, the poly(ethylene glycol)s, the PEO has minimal adverse reactions to skin (skin irritation and sensitization), as well as a sufficient loading capacity of drug dose. Unlike its low molecular weight analog like poly(ethylene glycol), which tends to form liquid or semisolids, PEO forms a solid matrix. The drug delivery property of the polymer electrolyte film for iontophoresis is assessed by checking its AC impedance.

PEO-salt complexes can be formed as soft, flexible films with a thickness that can vary from a few micrometers to about 100 micrometers. Previous studies showed that PEO can incorporate large concentrations (˜4M) of salt, making it eminently suitable as a matrix into which highly potent drugs may be incorporated.

Preparation of polymer-nicotine films: Films of PEO (RMM: 4,000,000, Aldrich) mixture are prepared by a standard solvent casting technique used for the preparation of polymer electrolyte films. The compositions are in the form PEOn:salt (where n=10 or 20). This represents the molar ratio of the ethylene oxide (EO) repeating unit to the salt. PEO10:salt represents 1 molecule of salt associated with 10 EO units. For each preparation, 1 g of PEO is used and the mass of salt to be used is calculated by dividing the molecular mass of the salt by the molar ratio of 10 and the molecular mass of EO repeat unit (i.e. 44).

The calculated mass of salt is then added to the 1 g of PEO in 50 mL of distilled water and stirred until complete dissolution. The mixture, which is a viscous solution, is then cast into polystyrene culture dishes (1-2 cm diameter). The solution is then covered and water is allowed to evaporate at a room temperature. The film is then peeled from the well and stored in a sealed plastic bag over silica gel in a desiccator.

The film can be tested by applying it to a cadaver skin sample mounted in the diffusion cell. The same scheme of pulse patterns can be used to determine delivery efficiency. The receptor compartment solution can be sampled and analyzed using EIA analysis and TLC to determine the electrochemical stability of nicotine using this delivery methodology.

What is claimed is:

1. An agent delivery device comprising:
an agent delivery reservoir containing an agent to be administered to a patient;
an electrolyte that is mixed with the agent to form electrolyte-agent mixture that is contained in the reservoir and traps the agent until electric current is applied;
an agent delivery surface in communication with the electrolyte-agent mixture, the agent delivery surface adapted to contact the patient and deliver agent received from the reservoir to the patient; and
a controller in communication with the electrolyte-agent mixture, the controller providing a series of controlled pulses to the electrolyte-agent mixture, each pulse allowing the device to administer a portion of the agent the patient, the series of pulses providing a temporally varying concentration of agent in the patient.

2. The device of claim 1 wherein the electrolyte comprises an iontophoretic electrically conductive material.

3. The device of claim 1 wherein the temporally varying concentration of agent includes a plurality alternating agent concentration maxima and minima, the maxima and minima differing by a predetermined amount.

4. The device of claim 3 wherein the temporally varying concentration of agent is matched to the turnover of cell receptors for the agent.

5. The device of claim 3 wherein the temporally varying concentration of agent is matched to the life-cycle of an invading bacteria or parasite

6. The device of claim 3 wherein the temporally varying concentration of agent is such that agent concentration maxima in the patient is increased over time.

7. The device of claim 3 wherein the temporally varying concentration of agent is such that agent concentration maxima in the patient is decreased over time.

8. The device of claim 1 wherein one or more intervals of the series of controlled pulse are varied over time via the controlling algorithm.

9. The device of claim 1 wherein the amplitudes of the series of controlled pulses are varied via the controlling algorithm.

10. The device of claim 1 wherein the duration or width of the series of controlled pulses are varied via the controlling algorithm.

11. The device of claim 1, wherein the controller includes a digital controller and a memory accessible to the digital controller.

12. The device of claim 11, wherein an algorithm for controlling the agent delivery is encoded in the memory, the algorithm being executable by the digital controller.

13. The device of claim 1 wherein further comprising a sensor system for determining the concentration of an agent in the patient.

14. The device of claim 13 wherein information from the sensor system is used to adjust the concentration of the agent or one more additional agents in the patient.

15. The device of claim 1, further comprising an attachment section for attaching the device to the patient.

16. The device of claim 15 wherein the attachment section comprises an adhesive surface.

17. The device of claim 1 further comprising one or more additional agent delivery surfaces.

18. The device of claim 1 wherein the agent delivery reservoir further contains a delivery enhancer that aids in delivering the agent to the patient.

19. The agent delivery system according to claim 1, wherein the agent delivery reservoir further contains a skin healing agent to prevent irritation caused by the agent delivery system.

20. The agent delivery system according to claim 1, wherein the agent delivery reservoir contains layers of compounds.

21. The agent delivery system according to claim 1, wherein the electrolyte comprises poly(ethylene oxide).

22. An agent delivery device comprising:
an agent delivery reservoir containing an agent to be administered to a patient;
an electrolyte that is mixed with the agent to form an electrolyte-agent mixture that is contained in the reservoir and traps the agent until electric current is applied;
an agent delivery surface in communication with the electrolyte-agent mixture, the agent delivery surface adapted to contact the patient and deliver agent received from the reservoir to the patent; and
a controller in communication with the electrolyte-agent mixture, the controller providing a series of control pulses to the electrolyte-agent mixture, each pulse allowing the delivery system to administer a portion of the agent to the patient, the series of pulses providing a temporally increasing concentration maxima of the agent in the patient for a first predetermined time period.

23. The device of claim 22, wherein the series of pulses provides a temporally decreasing concentration maxima of the agent in the patient for a second predetermined time period, the second predetermined time period occurring after the first time period.

24. The device of claim 22, further comprising an attachment section for attaching the device to the patient.
